(19) United States
(12) Patent Application Publication
(10) Pub. No.: US 2008/0145911 A1
(43) Pub. Date: Jun. 19, 2008

(54) Photocontrol of sugar redox reactions

(76) Inventor: Terrence S. McGrath, Longmont, CO (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/982,519

(22) Filed: Nov. 1, 2007

Related U.S. Application Data
(63) Continuation of application No. 10/683,384, filed on Oct. 10, 2003, now abandoned.
(60) Provisional application No. 60/417,781, filed on Oct. 11, 2002.

(51) Int. Cl. C12N 9/04

(52) U.S. Cl. 435/190

(57) ABSTRACT

A method is described for modifying a redox reaction using selected non-ionizing radiation. In a model reaction, oxidation of a sugar such as glucose in the presence of glucose oxidase can be completely inhibited with a non-coherent excited state oxygen spectral energy wavelength.

### REFERENCE TO RELATED APPLICATIONS

This application is a continuation of U.S. patent application Ser. No. 10/683,384 filed Oct. 10, 2003, which claims benefit of U.S. Provisional Application Ser. No. 60/417,781 filed Oct. 11, 2002, the contents of each being incorporated by reference in their entirety.

### BACKGROUND OF THE INVENTION

The invention includes a novel model of the atom that describes the underlying structure of particles and fields, yielding causal understanding and predictive tools for the formation and manipulation of elemental particles, atoms, chemical bonds, biological processes and photo-stimulation.

Early Theoretical Models—There have been numerous physical models of the atom since Lord Kelvin described it as a permanent vortex structure within the context of an ether background. J. J. Thompson improved the model with the discovery of electrons in 1897. Later the atomic model became known as the plum pudding model where the atom was pictured as holding negative electrons within a sphere of unknown non-electrical forces spread evenly throughout the atom (like raisins in plum pudding). The plum pudding model was also theorized to explain the different wavelengths of light based on the atom's size.

The pudding model was proven wrong based on experimental scattering data gathered by Rutherford almost 100 years ago. Rutherford showed that alpha particles slammed into thin gold foil sheets produced scattering only when the centerpoints collided and concluded that the entire mass of the atom is held at a finite center point. This supported the point nucleus theory and its infinitesimally small size in relation to the radius of the electron.

In 1913 Bohr suggested halo orbits for electrons, a model that explains quantum electrodynamics (QED) and electron angular momentum. This model, which shows the atom's electrons in orbit around a point-mass nucleus, is still popular today, although there are significant challenges, as it does not provide an accurate description of a point-mass center using just three dimensions. Einstein later proposed a three-dimensional space augmented with time as a fourth variable, or fourth dimension, in order to describe a space formed by a point mass in motion. This adjustment was required because a field could not be described without the point being in motion through time to create space and because four-dimension math better describes the structure of matter and fields.

Otherwise, for over 90 years the overall physical, or topological model for the atom has not changed substantially from a centerpoint-mass model despite significant advances in understanding the mathematical relationships of forces and particles within the atom and the discovery of a large number of particles that form the nucleus and constitute the strong and weak nuclear forces.

Physical Models—Most models that are used for educational purposes are designed to show the interlocking of molecular and chemical bonds with a variety of unique flanges. The minute scale of the centerpoint-mass nucleus relative to the electron orbit has made physical models difficult to portray, hence the focus on bonding models. Further, physical models have not portrayed the statistical models for the electron or an organizational construct for fields and the centerpoint mass.

Mathematical models—A number of theories have attempted to mathematically unify atomic forces. The present dominant model is commonly termed the “Standard Model”. The forces of the atom have been accurately described within the context of the Standard Model, where particles and force exchanges are represented in minute detail, matching experimental results. There are at least five major types of string theory that have unique base assumptions for gauge limits and dimensions (1 through 26 dimensions). String theories add time as a coordinate in unified space-time geometry. While three dimensions can describe a point, four-dimensions (three conventional plus time) are used to describe an event and a space. Logically extended, extra dimensions have been shown to describe forces and symmetrical constructs. Popular higher dimension theories have included four, five, ten and twenty-six dimensions. Through mathematical compactification, extra dimensions (>3) are “rolled up” to match our conventional three-dimensional world.

Several recent theories attempt to describe particles topologically, with the objective of: (1) providing boundaries and containment and (2) linking particles and forces more directly. Spin foams, twisters, M-branes, P-branes and D-branes mathematically describe particle forces that more closely represent a conventional view of objects that can spin, rotate, resonate and have volume. While they appear to provide a more accurate description of particles and force transfers, these theories do not describe the causal structure underlying the atom. Further, each of these mathematical models has to impose artificial limits to the math equations to account for the formation of the atom.

Mathematical models have flourished because the structure of symmetry, electromagnetic fields, charge, spin, confinement and gravity cannot be directly seen. The conventional view is that the atomic nucleus is a centerpoint mass and the vast space between the nucleus and the orbiting electrons is virtually empty. For almost 90 years, this has been considered by many as fundamental.

Current models do not establish the structure of the real and physical limits for regularizing fields and gauge limits as the center of the atom approaches zero. The models do not adequately accommodate the dynamic nature of the atom and therefore have limited ability to predict sub-atomic machinery and force interactions. The Standard Model describes mathematical relationships but is unable to locate a point in space at a given time. Relativity is not seen as relevant inside the orbit of the electrons. A new model of the atom is needed to combine the theories of the Standard Model and General Relativity to provide information in real time and space on bonding, force interactions and atomic substructures.

Deficiencies of the Known Models

Models have enhanced our understanding of Physics over the last century; however, each has had limitations in providing a grand unification theory. Bohr's model, for example, cannot account for other basic characteristics of the atom such as scattering or spectral absorption/emission from multi-electron atoms. The Standard Model and General Relativity as mathematical models have made significant contributions to the field of physics, but, despite these advancements, there has been little progress in tying these two descriptions of matter together. They differ dramatically in scale and mathematical complexity and they have not been unified.

Topological descriptions of particles provide some guidance for the structure of fields; however, what has remained elusive is a single physical model for the atom that provides the normalization and regularization factors that guide the formation of atoms and particles. Such a physical model should be based on a limited set of rules with minimal arbitrary elements and provide predictions of future events. A successful model should predict new experimental results and at the same time unify what has already been measured. A new model should also ideally provide lattice regularization for the formation of particles and provide lattice spacing that tends to zero at the centerpoint of the particle or atom. Further, the model should define limits of appropriate expectations of gauge-invariant observables.

To date, there has been no successful theory for the regularizations of the atom, that is, why atoms form in such consistent ways and in such tremendous numbers of iterations.

While mathematical models may accurately describe forces on the most basic levels, they have not yielded a plethora of experimental predictions going forward; nor are they able to describe the natural limits providing quantization of light, particle scales or atomic organization. Natural limits include the fundamental, real parameters for the formation of particles, light and atoms with such consistency and regularization. Natural limits would also define the “machinery” underlying the structure of fields, charge, photons and gravity. Further, it would yield constructive insights to the interaction of atoms within the context of chemistry and biology.

Another challenge to reaching a unified theory has been the significant scale disparity between the scale of force transfer and the scale of the proton. Strings are theorized to have force transfers starting on scales 20 orders of magnitude smaller than a proton. In some gauge theories, lattice volumes are described as zero, while other theories declare the smallest material dimension as a Planck length.

The wide variety of multi-dimensional theories makes a unification theory appear even more difficult to assemble. Popular string theories range from one to 26 dimensions. Force transfers are sometimes assigned particle values; sometimes particles are theorized with no dimension. Electron excitation can only be “explained” for hydrogen and has not been successful for many-electron atoms because the current model for hydrogen requires increasing radii for each energy level, an assumption that is unworkable in many-electron atoms.

A long-standing objective has been to unify gravity with the structure of matter. Most physics theories do not include computations for gravity, much less describe the mechanism for its generation. Current theories cannot explain the structural origin of fields or handedness (chirality) despite being able to measure both with high accuracy.

Current theories also do not postulate causality for discrete sizes of particles (the “hierarchal problem”). Symmetry is described mathematically, most often as positive and negative integer values, but current physical models do not explain a causal mechanism in the conventional realm for these values. No theory today answers the structure of mass gap, confinement, gravity, field generation or charge. Neutrinos remain an enigma. Black holes and large cosmological objects appear to follow another set of rules. The source of extra-gravitational forces in the universe (postulated as dark matter) is not understood. No theory explains the structural reason why inertial mass and gravity mass are the same. No theory provides a structural basis for the Pauli exclusion principal or Hund's rule. Although, many theories have offered significant insights into these questions, none has proven all-inclusive.

The important role of physics in biology and chemistry is often underemphasized. While bonds can be described mathematically, physics cannot describe the structural mechanism for bonding radii or the atomic-level coding that is locked in amino acids to differentiate genes and the life they generate. Grand unification theories seek a set of equations that describe all phenomena. No such model currently exists.

It is known that electromagnetic radiation can interact with atoms. The structure of atoms allows for the discrete absorption and emission of photons. This interaction is measured to very high accuracy; however, there is no predictive model of the machinery that causes absorption and emission. The trend has been to find uses for lasers of higher and higher power and apply them for shorter and shorter time periods.

Lasers and monochromatic light alter the behavior of atoms. In a recent experiment the atom was treated with a laser to hold the atom between energy states to make it super cold (Hau, Lene Vestergaard, “Frozen Light,” Scientific American, p 66-72, July 2001). The experiment applied one spectral frequency and reduced the speed of a second frequency of photons.

It is known in physics that monochromatic light has unusual effects on matter, and most of the last century has been spent building mathematical algorithms to explain phenomena such as excitation levels of atoms. Application of light wavelengths shorter than 400 nm to metal causes electrons to be emitted. Flash photolysis, under specific conditions, stimulates chemical bonding and lasers have been shown to change gold into mercury, but these effects do not have a predictive physical model to direct future discovery.

In nature, low-energy transfers of photon energy between cells have been shown to stimulate growth (Triglia, A. et al., “Biological Aspects of the Ultra Weak Photon Emission from Living Systems During Growth,” International Institute of Biophysics, Catania, Italy, 2001. Redox reactions within the cell have been stimulated using semi-conductor lasers as demonstrated by shifts in measured peak spectra as three different frequencies were applied successively: 820 nm, 670 nm, and 632.8 nm (Karu, T. I. et al., Changes in Absorbance of Monolayer of Living Cells Induced by Laser Radiation at 630, 670 and 820 nm, Journal on Selected Topics in Quantum Electronics, Vol. 7, no. 6, November/December 2001; Karu, T. I. et al., Irradiation with a Diode at 820 nm Induces Changes in Circular Dichroism Spectra (250-780 nm) of Living Cells, Journal on Selected Topics in Quantum Electronics, Vol. 7, no. 6, November/December 2001). Optical measurements of absorbance changes in mitochondria were taken and showed that increased electron transfer occurred. Other experiments have shown too much intensity in too short a time period causes heat and is counter productive. Some specific wavelengths at low-power applications are used for beneficial effects such as stimulating cardiac tissue; however, universally the mechanism is not understood and therefore cannot be leveraged to predict and therefore maximize the beneficial potential of such treatments.

In redox reactions, the quenching of excited state molecules by lower state molecules is well studied, but it is not well understood because there is no predictive model of the machinery of elemental interaction or the exchange of elemental energy within a bond. A theory by Minaev (Weldon, Dean et al., “Singlet Sigma: The “Other” Singlet Oxygen in Solution,” Photochemistry and Photobiology, 1999, 70 (4), 369-379) suggests that spin-orbit interactions can provide a means to steal intensity from higher energy states but no mechanism is provided. A complete atomic model should be able to describe such interactions.

The applications of energy in biostimulation at high power levels, adding too much energy through biostimulation is counter-productive.

Another major question concerns the nature of a dimension. Mathematically, dimensions and complexity are simply positive, negative, real or imaginary numbers. A multi-dimension model that involves tangible structure for dimensions should render the structure of matter and forces to be real, and although complex, they should be determinable and not subject to uncertainties and probabilities. A successful physical atomic model should translate a dimension into conventional terms, yielding a plethora of predictions based on the model itself.

Current models also do not establish the structure of the real and physical limits for regularizing fields and gauge limits as the center of the atom approaches zero. The models do not adequately accommodate the dynamic nature of the atom and therefore have limited ability to predict sub-atomic machinery and force interactions. The Standard Model describes mathematical relationships but is unable to locate a point in space at a given time. Relativity is not seen as relevant inside the orbit of the electrons.

A new model of the atom that combines the Standard Model and General Relativity to provide information in real time and space on bonding, force interactions and atomic substructures would be of significant value in providing a detailed representation of atomic structure and allowing development of methods to modify chemical reactions and bonding strength.

### SUMMARY OF THE INVENTION

The Axial Model, herein also referred to as the Model, is based on a six-choose-four permutational metric where trapped energy is sequentially transferred through four-dimension spaces within the six-dimensional atom. The six-choose-four structure allows sets of four dimension spaces to form within the context of the atom's six total dimensions. These set of four-dimension spaces define what is called a metric set, or a symmetrical set of spaces that describes the real (non-negative) distances between neighboring points within the atom. The axial metric set naturally defines a centerpoint and a 15-axis lattice structure that can sweep about the centerpoint, rotate and create infinite fields while maintaining lattice spacing. The Axial Model ultimately defines 19 regularizations of the atom that define and limit the natural generation of forces and fields.

The problem of discovering the reason for the discrete hierarchal scale of particles and sub-particles is in the atom. The Axial Model uses radii and lattice count for high-density circle lattice sets as the radii for spindle torus structures that naturally form within triplet sets of six-choose-four axes. The spindle torus is a doughnut structure where the hole is missing, and the doughnut overlaps itself by as much as 90%.

Trapped energy is transferred within these discrete formations, importantly, defining the scale and structure of fields, moment, charge, chirality, and the structure of electrons versus protons. The Model also describes the natural structural reasons for particle scales with no compromises or missed steps from the proton down to the single lattice point, a scale of 4.69E-21 versus the proton in 6-D, consistent with the scale described by string theories.

The structure for atomic symmetry is also provided through use of axial triplets. Particle structures are described within the context of a 6-dimensional centerpoint with energy traveling through closed loops of space defined within a spindle torus structure where the torus scale is defined by naturally occurring high-density lattice circle set solutions. Mass gap and confinement are described by particles sharing lattice points. Photons are shown to be produced by the closed loops of energy within each particle with mass.

The Model provides the natural limits for the tightening metric and the 5-D deterministic orbits of electrons. Finally, the Model provides the structure and scale of the gravitation versus the electromagnetic fields in the range of 10E-39. The Model includes the multiple substructures of the electron, as well as additional substructures to the proton's quarks and pentaquarks.

The Model introduces a new concept within the atom called “resident energy,” which has significant implications for all sciences. Resident energy is the continuous flow of 4-D energy within the atom through defined geometry and periodicity that is responsible for the formation of electromagnetic fields, symmetry, charge, photons, radioactivity, chirality, mass gap, confinement, all particle scales and gravity. This fundamental energy is guided by only a handful of simple rules within the six-dimension metric. The Model reveals the tools to manipulate resident energy.

In chemistry, resident energy is defined within the context of bonds. The Model reveals that energy is held within the atom in a four-dimension context, independent of measurements of mass and that the atom is the repository of energy required for field generation, bonding, short-term excitation states and long-term resident energy levels.

Importantly, the Axial Model has utilities providing a large number of beneficial applications ranging from medicine to computers. The Model reveals the underlying mechanism for resident energy within atoms and provides tools for determining the proactive changes that can be made to the atom. Expected material outcomes can be predicted; and predict the material outcomes to expect. For example, the Model includes how to manipulate the energy and field structure for atoms within DNA through use of low energy and intensity electromagnetic energy.

Additionally, the Axial Model proposes a structure for gravity that is consistent with the scale and properties of gravity as they have been measured experimentally. The Model provides this structure within the context of the six-choose-four structure, thereby leading to a unified understanding of gravity and matter.

The Model contains a limited series of natural physical and geometric structures that provide the underlying foundation of the atom. The Model can be fundamentally understood with a minimal number of assumptions based on three key math sets described herein: (1) a spindle torus representing the particle structure (an overlapping doughnut with no hole), (2) Circle lattice equation for determining the number of lattice points on a circle to determine lattice radii and 3) Julia fractal equations for determining the transfer of energy within the lattice sets.

The Axial Model provides the fundamental structure to the organization of the atom. The Model provides a physical description of the atom as a geometric construct that is a visually intuitive description of the structure and position of particles, sub-particles, fields, photons and forces within the atom. This Model was designed to provide visualization of the tangible structure of the atom. The Model also is the basis for a variety of useful applications in medicine and computing through manipulation of resident energy within atoms using low energy electromagnetic waves.

### BRIEF DESCRIPTION OF THE DRAWINGS

FIG. 1 is a view in perspective of six directions of background waves converging to form a six-dimension (6-D) centerpoint according to the model of the present invention. Letters A through F represent six independent directions converging towards the six-dimension centerpoint G. The intersection of wave directions A and B form a hypertube H.

FIG. 2 is a view in perspective of the axial model of the present invention showing 15 “six choose four” or 6/4 axes each having a unique four-dimension lattice set (crests, troughs and null space included). Axis A is comprised of sets of four dimensions (e.g., abcd) chosen from six directions in the metric (abcdef). Axis B is comprised of four dimension sets (abcf). Axis C uses another four-dimension set (abdf). The 6/4 axes converge through the centerpoint D. The symmetry of triangle E is the similar to the symmetry of triangle F, although the relationship is inverted after the axes cross the centerpoint D.

FIG. 3 is a simplified axial triplet in perspective according to the present invention using three of 6/4 axes shown in FIG. 2. The three axes ABC converge through the centerpoint D. Applying the sequence 1, 2, 3 to both sides of the triplet provides rotation on one side of the centerpoint as equal and opposite the other side of the centerpoint, including the electron pairs opposite spin-up (positive) and spin-down (negative) character.

FIG. 4 is a view in perspective of the fifteen 6/4 axes shown in FIG. 2 further developed as 10 cones (five 6/4 triplets) that share a common center point each with one cone on opposite sides of the centerpoint shown in FIGS. 1 and 2. The triplet again defines spin up A versus spin down B. The drawing also show the interplay of the ten cones and the relative spin character for contiguous triplets D. Letter C show the conceptual equator, exaggerated.

FIG. 5 is a Julia fractal diagrammatic representation of a six-choose-four completion path, shown by direction and four dimension seiche sets, On the left hand column, directions ABCDF are represented as wave functions, Across the top of the diagram seiche sets from the triplet are represented. The column H shows that four directions are involved in the seiche ABCD. In column I a different four directions are involved ABCF as energy is transferred from H to I. Letter J shows the wave function for direction A which is involved in every seiche in this example, while K shows direction C involved in two of three seiche sets. Letter L shows the advanced/retarded wave of direction F. Letter M represents the Julia fractal (connected) where energy can be held, while N represents a disconnected Julia fractal representative of energy leaving or entering the 4-D space.

FIG. 6 is a schematic view of energy transfer between two crests points in the ABCD lattice, letter A, requiring energy transfers between two other lattice sets, ABCF represented by letter B and ABDF represented by letter C. The right figure represents a crests D, null spaces E and troughs F in the context of a hypertube field associated with one 6/4 lattice set.

FIG. 7 shows three representative lattice sets that provide natural limits for relative particle hierarchy scales. Letter A represents a circle radius of four with four lattice points on the circle, B represents a high-density radius of five with 12 points on the circle and C represents a circle radius of six with only four points on the circle.

FIG. 8 shows one of three 5-D completion paths in each particle each using three 6/4 lattice sets (from an electron quark, radius 25, seiche count 20). Letters A, B and C each represent one 6/4 set. Letter D represents the maximal distance that energy can transfer successfully between seiches.

FIG. 9 is a view in perspective showing three completion paths of the type shown in FIG. 8 forming a spindle torus. Letter A refers to one completion path. Letter B represents an exaggerated view of the intersection of the three paths straddling the radical helicoid, forming a Reuleaux shape. Letter C represents a side view of the three paths.

FIG. 10 is a schematic representation of two completion paths within the spindle torus according to the Model of the present invention The example shown represents an electron spindle torus D with radius 85, seiche count 36 and three electron quarks E, radius 25, seiche count 20. Letter F highlights the lemon of the torus, Letter R is the radius of the outside of the torus tube from the centerline, r is the radius of the inside of the tube, c is the distance from the centerline of the torus to the center of the tube, Z is the radical center of the torus and G is the intersection of the completion paths at the end of the spindle torus lemon.

FIG. 11 is a view in perspective of the hydrogen proton and electron modeled according to the present invention. Letter A renders the electron, B highlights the outside of the torus, C highlights the position and scale of the quarks and D highlights the torus lemon.

FIG. 12 is a view in perspective of the sequence and tilt of the rotating completion path planes create the radical helicoids according to the present invention. Letters A and B Represent the left-handed and right-handed tilt of the completion paths, respectively. Letters C and D represent the rotation of the field based on the tilt of the completion paths. Letters E and F highlight the auger-shaped radical helicoid generated by the three completion paths acting as rotating planes as a consequence of the tilt of the completion paths and completion path intersections straddling the radical axis.

FIG. 13 is a view in perspective showing 6/4 axial and mirror symmetry. Letters A and B represent the opposite spin of the triplet on either side of the centerpoint. Letters C and D highlight two neutrons exhibiting axial symmetry, sharing the centerpoint and having opposite spin while maintaining 1, 2, 3 sequence. Letters E and F represent the neutron and proton, respectively, exhibiting mirror symmetry on the same side of the centerpoint. Letters G and H highlight the opposite tilt of the completion paths between contiguous mirror symmetric particles.

FIG. 14 shows schematic representations of inter- and Intra-mass gap and shared seiches. Letters A, B and C represent tilted completion paths intersecting at the end of the spindle torus lemon, straddling the radical helicoid D within a single particle. Letter E represents the intra-mass gap shared seiche between intersecting completion paths. Letters F, G and H represent the neutron, proton and electron, respectively. Letter I represents the direction of energy transfer within the completion paths for each particle. Letters J, K and L represent shared seiches between contiguous mass particles within a triplet.

FIG. 15 is a view in perspective using two of the three completion paths and corresponding schematic sectional views illustrating Complementary Rotation and Confinement. Letter A shows the structure of a quark with radius of 325 and 60 lattice points with five pentaquarks with radius 65 and 36 lattice points. Letters G and H represent the opposite tilts of completion paths within contiguous particles with letter I representing the tilt of the completion path for the Quark matches the tilt of the outermost pentaquarks. The overlap of the circles of the minor confinement torus structure is about 90%. Letter B shows the structure of a proton with radius of 1105 and 108 lattice points, confining three quarks. Letter D highlights that the tilt of the outermost sub-particles must agree with the tilt of the confining particle. Letters E and F represent more fully developed spindle torus structures. The overlap of major confinement structures is about 65%.

FIG. 16 is a schematic representation of the intersection of shared seiches between particles and sub-particles. In the case of major confinement, letter A represents the completion path of a proton, letter B represents the completion path of a quark, letter C shows the point at which the particles share a seiche. In the case of minor confinement, letter D refers to the completion path of the quark, letter E refers to the completion path of the pentaquark and letter F represents the shared seiche between the particles. Letter G shows the position of the pentaquark seiche that is not confined by the quark structure, facilitating the rapid deterioration of an unconfined quark.

FIG. 17 is a schematic representation of the shared seiche position of the electron relative to a proton. Letter A represents the completion path of the proton, letter B represents the path of the quark and letter C represents the position of the electron.

FIG. 18 is a schematic representation of particle hierarchy scales within a single lattice scale. The model represents the following particles from a single seiche to the scale of a proton, including the “r” radius of the spindle torus and respective lattice seiche count. Major confinement particles are electrons and protons. All particles with a represented five-particle substructure are minor confinement particles.

| | | # Lattice Points |
|---|---|---|
| | | on High-Density |
| Particle | Radius | Circles |
| Seiche | 0.5 | 1 |
| Sub-pentaelectron | 1 | 4 |
| Pentaelectron | 5 | 12 |
| Sub-Pentaquark | 13 | 12 |
| Electron Quark | 25 | 20 |
| Pentaquark | 65 | 36 |
| Electron | 85 | 36 |
| Quark | 325 | 60 |
| Proton | 1105 | 108 |

FIG. 19 is a schematic view showing the axial alignment of the neutron and proton. The axial structure of the centerpoint, neutron, proton and electron are represented by letters A, B, C, and D, respectively. Letters E and F represent the direction of energy flow in the completion path through the lemon of neutrons and protons, respectively. Letter G represents the path of flow for the outside of the torus responsible for field generation. Letter H represents the conceptual plane where the periodicity of the proton and neutron mirror flow meet. Letters I, J and K represent more fully developed spindle torus structures.

FIG. 20 is a schematic view, showing the structure of charge for a neutron or proton. Letter A represents the direction of energy transfer within the sequential completion path. Letter B represents the direction of flow along the radical helicoid. Letters C and D represent the attractive (positive) and repulsive (negative) charges located at the endpoints of the lemon. Attractive and repulsive fields associated with a particle are generated by the direction of energy flow in the particle's completion paths.

FIG. 21 is a schematic view, showing the structure of the electromagnetic field. The centerpoint A and individual directions are the local generator of the electromagnetic field. Points of 4-D occupiable space are continuously generated spontaneously and sequentially by the intersection of four directions B and a slice of such a field can be conceptually represented by a fibonacci seedhead©. The patterns associated with the high-density completion paths can be visualized by the spirals generated by the seedhead structure.

FIG. 22: is a Julia fractal diagrammatic representation of a photon, shown by direction and four dimension seiche sets. On the left hand column, directions ABCDF are represented as wave functions. Across the top of the diagram seiche sets from the triplet are represented. Letter G highlights that there are still four dimensions included in the formation of each seiche, however, one of the wave functions has lost its periodicity and now transfers unencumbered from seiche-to-seiche at the speed of light, as represented by letter H. The remaining directions BCDF maintain the same periodicity they had within the particle completion path from which the photon was emitted.

FIG. 23 is a diagrammatic representation of a triplet cone as additional neutron/proton/electron sets are added in discrete scales according to the Model. Letter A represents the spin direction (sequence 1, 2, 3) of a level 1 particle. Letter B highlights that at level 2, the particle sequences in an opposite direction (sequence 2, 3, 1^{prime}) from level one. Letter C shows that as the three particles are added in level 2, each is larger than the former in discrete scales described by the model. Letter D and E show the same rules for particle additions apply to level 3 and 4 also. Level 2 adds up to three additional particle sets to the base cone. Level 3 adds nine more potential particles. Level 4 (unstable) adds potentially 27 more. Letters F and G refer to inside and outside positions on a cone, which affects the observed electron orbit for those positions.

FIG. 24 is a diagrammatic representation of the electron cloud according to the Axial Model. Letters A, B, C and D refer to the centerpoint, neutron, proton and electron, respectively. Letter E refers to the 5-D determinable positions associated with the electron (visualized only in 3-D) as a resultant of the 6/4 triplet particle structure using three sets of four-dimensional spaces to create 5-D particles.

FIG. 25: is a diagrammatic representation of electron orbits within the 6/4 axial field and triplet cone structures according to the Model. The model represents the 5 triplets within the atom as the location for the electron pairs. The triplets account for the x, y and z factors generally measured in the electron orbit. Letter A highlights the position of the axis associated with the X-axis. The orbital positions of the remaining axis are shown below. Opposite spin pairs are located at the other end of the triplet. Letter B highlights the conceptual equator and letter C shows measured electron orbits. The model shows some orbits may be influenced by relative positions on the inside or outside of the cone.

| Triplet | Orbit |
|---|---|
| 1 (X axis) | 1s, 3s, 5s, 6s |
| 2 (Y axis) | 2s, 3d_{z}, 3d, 4d |
| 3 (Z axis) | 2p, 3p, 3d, 3d, 4d |
| 4 (W axis) | 2p, 3p, 3d, 3d, 4d |
| 5 (U axis) | 2p, 3p, 3d, 3d, 4d |

FIG. 26 is a view in perspective of the axial particle structure of select elements according to the present invention. Letter A highlights the Hydrogen atom, letter B highlights the simple structure for helium. Letter C renders the asymmetric model for Lithium7. Letter D show the paramagnetic structure for doublet oxygen and letter E shows the balanced structure for neon.

FIG. 27 is a diagrammatic representation of a particles relative position to other particles in the context of the atomic levels as described in FIG. 20 according to the Model. Letter A shows the orientation of the five triplet sets and the atom's conceptual equator. Letter B renders the sequence of position and spin direction for neutron and proton particles relative to the ten base cones, consistent with Hund's rules. Letter C highlights the completion of level 1, letter D highlight the structure of Argon in the context of level 2.

FIG. 28 is a diagrammatic representation of the generation of gravity in the context of the electromagnetic field according to the Model. Gravity waves are generated by the three finite completion paths within a finite portion of the electromagnetic field shown as letters A, B and C for the neutron, proton and electron completion paths, respectively. The electromagnetic field D is generated by the six directions at the centerpoint E.

### DETAILED DESCRIPTION OF PREFERRED EMBODIMENTS

The following definitions are used herein.
Atom—A six-dimensional structure with particles in five dimensions and energy transfer in four dimensions. The reference to an atom is not limited to a single atom and can refer to small and large groups of atoms.
Atomic equator—The structural division of the atom where five cones are on one side and five cones are on the other side of the atom, orthogonal to the helium triplet.
Axial metric—The structure of the space created when six hyperwaves converge to a single point creating four-dimensional spaces of determinable distance and symmetry using 15 axes of the four-dimension spaces and the six-dimensional centerpoint.
Axial Model—The title of the Model that includes the 19 regularizations or natural structures that account for the structure and consistent duplication of matter.
Axial triplet—The basic structure formed by three 6/4 axes that provides the structure for particle formation.
Charge—The attractive and repulsive flow of energy associated with the sequential cyclic flow of energy disturbing the background within the spindle torus particle and completion paths.
Chiral Field—The field generated by each particle completion set in the context of the electromagnetic field that naturally twists as a result of the three tilted completion paths straddling the radical helicoid. It is caused by the triplet energy transfer associated also with charge, magnetic moment and gravity.
Completion path—The continuous path of trapped energy within a particle that cyclically and sequentially transfers through three high-density lattice point sets and shared seiches, back to its original starting position. It is constructed using 3 sets of 4-D lattice seiches creating a 5-D path.
Completion set—Three completion paths that make up a particle.
Cone—An axial triplet on one side of the centerpoint in which up to 13 particle sets of neutrons, protons and electrons can form.
Cone pair cleaving—The release of cone pairs (based on groups of axial triplets) from the atom because the triplet-based cone pair has lost timing with the centerpoint and the sequence of the atom. Usually a function of the centerpoint being moved abruptly.
Confinement, major—A particle set based on having an internal structure comprised of three sub-particles, each ˜2.5% of the mass of the confining particle. Electrons and Protons exhibit major confinement.
Confinement, minor—A particle set based on having an internal structure comprised of five sub-particles, each ˜0.8% of the mass of the confining particle. Pentaquarks and electron quarks exhibit minor confinement.
Dimension or direction—A real variable (“D”) used to identify a position in space at a given time. Six dimensions or variables identify the location of the centerpoint. Four directions are required to define the location of an occupiable space. The mass hyperplane waves provide the original directional energy. Once formed, the centerpoint organizes the local electromagnetic field.
Electromagnetic field—The field of four-dimensional potentiated spaces formed within the six-dimensional metric out to infinite scales. It is generated from the centerpoint. Potentiated spaces are occupiable four-dimension points that may or may not transfer energy. They contribute to metric organization.
Flow—The continuous transfer of energy from seiche to seiche within the completion paths. Energy transfers between seiches at the speed of light, once seiches are formed sequentially. It is instrumental in the propagation of field structure, helicity, charge and gravity waves.
Field strength—The strength of the electromagnetic and gravity fields and the organizational potential of the field of 6/4 seiches. Field strength is increase with smaller and tighter seiches and an increased amount of energy bound within the completion paths associated with metric tightening.
Gravity—The background disturbance created by the synchronous flow of trapped energy within a particle/atom through completion sets, outward from the centerpoint and then returning inward, back to the centerpoint. The cyclical flow path disturbs the background of space in waves.
Helicoid—The auger-like shape of the radical axis within the axial triplet and spindle torus particle created by the three tilted completion paths.
Hyperplane waves—The waves of potential energy in the background of space and are not visible conventionally. They are made up of gravitational force, the electromagnetic field generated by the six directions and photons.
Hypertube—The intersection of two opposed sets of hyperwaves to create a concentration of background energy.
Information paradox—The conversion of chaotic mass to massive completion sets of trapped energy associated with black hole formation.
Light—Light is designated to be electromagnetic waves of any wavelength across the entire electromagnetic spectrum.
Mass—A particle that has a sub-structure of trapped photon energy flowing in closed loops called completion paths. Mass is measured in three dimensions and is a function of the scale of high-density lattice point completion paths emanating outward from the atomic centerpoint. To be considered mass, the particle must have energy flowing through completion paths.
Mass gap—The loss of apparent mass between contiguous particles as the atom gets larger. Mass gap is a manifestation of particles sharing spaces. Mass gap occurs between contiguous triplet particles and within the atom when particle completion paths cross.
Material—Related to mass.
Maximal distance—The longest distance between seiches where energy can be transferred successfully. This distance is the same magnitude as Planck length.
Metric set—A metric set is a group of spaces that can be described by the real (non-negative) distances between neighboring points in a set that is also symmetric.
Metric tightening—The reduction of the relative radius of a particle by adding energy to a particle so that additional completion paths can be formed.
Neutron—A major confinement particle of 108 lattice seiches per completion path and contains five dimensions. It shares the centerpoint. It has the exact structure of a proton.
Neutrino—A single six-dimensional point, often located at the center of the atom. It is the propagating starting point for the formation of matter associated with all mass particles.
Obstruction—A force that disrupts the transfer of energy from seiche to seiche, at minor energy levels changing the direction of a completion path, at larger levels causing completion path trapped energy to stop.
Particle—A five-dimensional spindle torus structure organized within three 6/4 axes. Particles have flow and mass.
Orphan wavelengths—Individual wavelengths associated with an atom that are isolated from major groups of that particle's wavelengths, ideally, by at least 7-10 nm.
Photon—A set of five-dimension energy emanating from a single completion path where four of the waves remain periodic, consistent with the originating particle, and the fifth wave has lost its periodicity, allowing the particle to move at the speed of light.
Proton—A major confinement particle of 108 lattice seiches 6/4 lattice set per completion path (total of 972 seiches per proton).
Power—The quality of a hyperwave and hyperwave intersections determined by the frequency, amplitude and tightness of the waves. Waves equal in power and phase alignment have the highest level of interaction.
Radical Axis—The auger shaped centerline of the spindle torus that is the generated by the tilted, rotating planes associated with completion paths. Radical is defined as the mean distance from each of the triplet axes. A triplet centerline.
Regularization—The natural reason for atoms to form consistently and in such tremendous numbers of iterations.
Relative radius—The quantization of the scale of particles within atoms based on metric tightening.
Resident energy—The energy in four, five, and six dimensions that moves in and out of the particle without changing the particle's measurable mass.
Reuleaux lemon—The center structure of the spindle torus particle formed by the intersection of three rotating planes of trapped energy flow.
Seiche—The smallest space occupiable by energy. It is a four-dimension space generated by sequential rotation of five or six dimensional variables within an atom.
Seiche density—The factor of the seiche count divided by the radius of the particle.
Secondary wavelengths—Spectral wavelengths of particles that have already undergone metric tightening. All wavelengths except the highest spectral intensity for the atom.
Six-choose-two (6/2)—The interaction of two background waves that forms a hypertube whose character is dependent on the quality of the interaction (power, direction).
Six-choose-four (6/4)—Within a six-dimensional structure that forms the foundation for the atom, there are permutational sets of four-dimensional spaces that are formed through which energy transfers. The permutations yield 15 axes of four-dimensional spaces, including crests, troughs and null space, converging through the six-dimensional centerpoint.
Six-choose-six (6/6)—The single intersection point (centerpoint) associated with the formation of the atom when six hyperplane waves of equal power converge.
Spontaneous and sequential—The formation and dissolution of 4-D potentiated spaces in the electromagnetic field as generated from the 6-D neutrino and sweeping about the centerpoint.
Sweep—The rotation of the metric can be based on (1) a single direction rotating about the centerpoint, influencing the sequential formation of seiche points, or (2) multiple directions (up to six) rotating simultaneously about the centerpoint.
Symmetry, axial—Symmetry between contiguous particles that share seiches on the opposite sides of the centerpoint within an axial triplet set.
Symmetry, mirror—Symmetry between contiguous particles that share seiches on the same side of the centerpoint.
Trapped energy—The energy of photons flowing in a cyclic closed loop within a particle through a number of high-density lattice points.

The model provides 19 regularizations that describe how atomic patterns are duplicated. The regularizing factors include:

1. The Six-Choose-Four Permutational Metric (6/4) with Four-Dimension Lattice

2. 6/4-D Axial Triplets, Ten Cones and Five Radical Axes

3. The Background of Space

4. The Interaction of Hyperplane Waves

5. Four-Dimension Energy Transfer

6. Flow Through High-Density Lattice Points and Circle Completion Paths

7. The Spindle Torus Particle and Resident Energy

8. The Radical Helicoid

9. Axial and Mirror Symmetry

10. Mass Gap and Shared Seiches

11. Confinement—Major and Minor

12. Discrete Particle Sizes

13. The Proton and Neutron

14. The Photon

15. Expansion of Ten Primary Cones

16. More Magic Numbers for Protons and Neutrons

17. Electron Orbits

18. Tightening Metric

19. Gravity

The Six-Choose-Four Permutational Axial Metric (6/4) with Four-Dimension Lattice

As a starting point it is assumed that energy transfer in space is on a 4-D basis, consistent with four dimensions used in the Standard Model and Special Relativity. It is also assumed that a dimension has to have some properties that affect the formation of matter, something “real” as opposed to being an imaginary mathematical integer. Many forces move as waves, so it is postulated that a dimension might be thought of as a wave of energy, of some type, in the background. It is also postulated that there is a construct that focuses matter formation at a point. Converging sound waves can create standing waves of constructive and destructive interference, so it is postulated by analogy that complex energy waves (defined as hyperplane waves) in the background converge to potentiate conventional matter and space. It is also assumed that matter, once formed, had to be sustained locally as discussed in Baez, John C., “Higher-Dimensional Algebra and Planck-Scale Physics,” in Physics Meets Philosophy at the Planck Length, Eds. Craig Callender and Nick Huggett, Cambridge University Press, Cambridge, 1999.

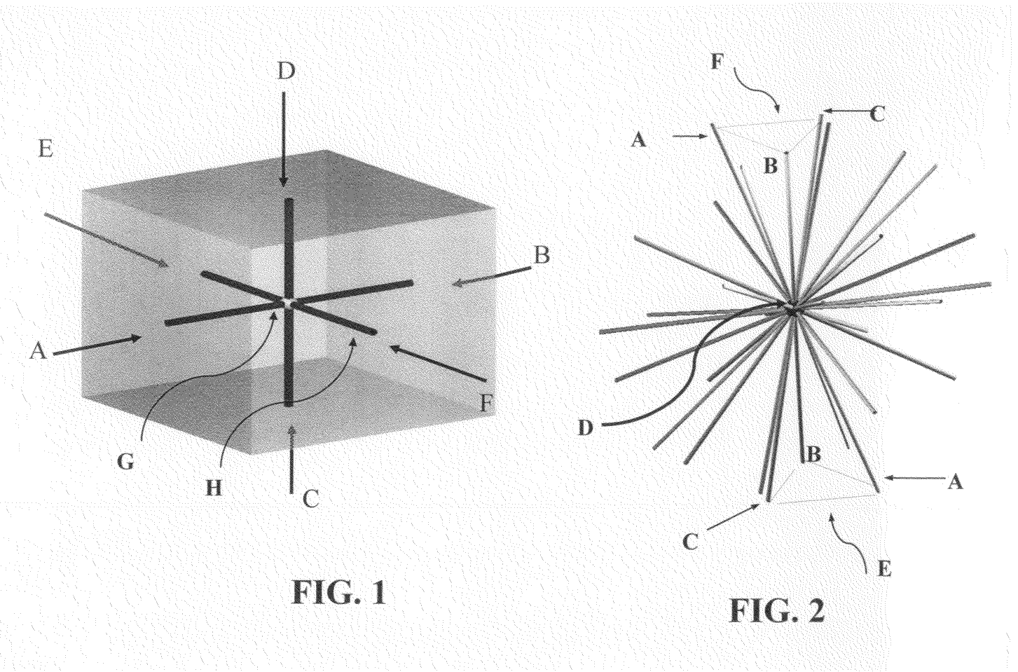

Six dimensions (or directions of background waves) intersecting at a six-dimensional centerpoint can naturally create a real four-dimension axial lattice based on permutational sets of four direction intersections (FIG. 1). The six-dimension Axial Model (FIG. 2) incorporates 15 axes of four-dimensional spaces organized through a single six-dimensional centerpoint (under ideal conditions, separated by equal angles of arcsine 1/3 in r⁶). The mathematical description for such a permutational metric is defined in the Model as “six-choose-four” (6/4).

Sets of four-dimension spaces are chosen from among the six possible dimensions (Table 1). Directions and dimensions are used interchangeably within the paper, as they are the same. It takes 6 variables to define the centerpoint and it takes 4 variables to define each potential real space occupiable by energy. FIG. 1 shows the directions coming from opposite directions, aligned to match three dimensions using axes X, Y and Z. This drawing is idealized with a 3-D appearance, however, and the actual directions are rarely directly opposed, adding character and sweeping to the metric.

TABLE 1
Six-Choose-Four Axis Permutation Sets

| | |
|---|---|
| | (1) |
| $C ( m , n ) = \frac{m !}{( n ! ) ( m - n ) !}$ | |
| $C ( 6 , 4 ) = \frac{6 * 5 * 4 * 3 * 2 * 1}{( 4 * 3 * 2 * 1 ) ( 2 * 1 )} = 15 axes 4 - D sets$ | |

Each 4-D axis is made up of specific permutational sets of four-dimensional spaces aligned on axes that pass through the centerpoint. Each of the axes represents a natural organization of the atomic structure. While there is no specific order or starting point, an example of the 15 six-choose-four axes is shown in FIG. 2 and Table 2. Each of the six-choose-four axes is made up of crests and troughs of 4-D occupiable space with non-occupiable null space between the seiches providing lattice spacing. The structure of the 6/4 metric with the crest and trough wave interactions clearly defines a field that can be tightened but resists the forces to be compacted to zero as they approach the single centerpoint. The Model defines the centerpoint at a specific point in space at a specific time without artificial limits. The Model also defines the lattice of 4-D spaces in the surrounding field.

TABLE 2
15 Axes of
Six Choose Four Spaces
Sets of four-dimension points chosen
from a set of six directions: ABCDEF

| | | | |
|---|---|---|---|
| GROUP 1 | ABCD | ABCF | ACDF |
| GROUP 2 | ABEF | ABDF | ABDE |
| GROUP 3 | ABCE | ACEF | BCEF |
| GROUP 4 | ADEF | ACDE | CDEF |
| GROUP 5 | BDEF | BCDF | BCDE |

A four-dimension point or node within the 6/4 lattices is defined in the Model as a “seiche” (pronounced sēch, similar to “teach”) a nautical term for the spontaneous intersection of resonant waves, usually in a lake. While the term “node” is almost always used to describe lattice intersections, it implies more of a fixed relative position. In the Model, each direction/wave moves about the centerpoint independently, creating a periodicity to the spontaneous formation and dissolution of 4-D intersection points. Change any single direction's strength, and periodicity, angle and the entire atomic system will start to shift. The centerpoint, once formed, is the propagator and sustainer of the local 6/4 metric for each atom. It is also the originating source for the electromagnetic field. The six-choose-four lattice organization also creates infinite fields and yet provides lattice regularization limits down to the centerpoint. As one 4-D axis hinges and sweeps about the 6-D centerpoint, all of the 4-D axes move. A six-choose-four axial metric naturally organizes fields and particles within complex space. The centerpoint is constantly renewed by the local background waves. In the absence of local background waves associated with gravity and electromagnetic fields (e.g., deep space), particles lose resident energy, resulting in weakened fields, failed bonding and diminished atomic level interactions with other elements (e.g., calcium loss by astronauts).

The atom's initial formation can be conceptualized as similar to a set of standing waves, except that once the centerpoint of the atom is formed and traps photon energy the atom's grid propagates locally, from and through the six-dimensional/directional centerpoint. The dimensions are six real wave interactions (versus mathematically complex 12 dimensions) and create positive space outward from the centerpoint.

A visual analogy of the intersection of six waves can be constructed using a cubic box with six flat speakers (one on each side pointing inward). Standing waves are created with the interaction of two matched, opposed speaker waves tuned to match power levels (frequency, amplitude and direction). Each resulting standing wave is either constructive (crest) or destructive (trough) separated by null space.

Rotation and changes to the atom are influenced by the atom's self-generated self-referencing structure and by outside influences. Each hyperplane wave direction influences the atom. Non-orthogonal alignment of the six directions improves the atom's rotational sweeping movement about the six-dimension centerpoint. As one 6/4 axis sweeps, all other 6/4 axes move, creating a “gearing” effect, hinging at the centerpoint. For simplicity, the Model is idealized with all six directions intersecting at right angles, while in nature, this perfect equilibrium occurs rarely.

Scattering—Complex 6/4 matter organized around a 6-D centerpoint is locally and axially self-referencing and therefore does not interact significantly with other 6/4 matter or energy (photons pass through each other). On the other hand, six-choose-six (6/6) dimensional centerpoints are not self-referencing (because they have no trapped energy flow, as discussed later) and will interact (scatter) only when hit with other 6/6 (or more complex) centerpoints. All forces continue to be measured through the centerpoint, consistent with Rutherford's experimental findings. Rutherford defined all mass as being actually located at the centerpoint rather than measured through the 6-D centerpoint, as defined by the disclosed Model. The Axial Model does not affect Rutherford's experimental findings, but it does lead to predictions and information not available from Rutherford style scattering experiments and the centerpoint mass Model.

Four-dimensional space is not simply adding one variable to the conventional 3-D view of the world; rather, it consists of sets of four independent dimensions that do not conform to three-dimensional visualization. While this is intuitively consistent with Einstein's math of three dimensions+time (4 variables) and is consistent with experimental measurements, it is not obvious. It has recently been conjectured that 4-D space may be closer to describing reality than current theories, yet it is understood that a 4-D space/particle construct would not be visible conventionally.

The Model predicts that when a 6-D centerpoint hits another 6-D centerpoint is the timing of the 4-D field flow disrupted and scattering data yielded. The Model also shows that the space between the centerpoint and the orbiting electron is filled with trapped energy and potentiated 4-D spaces. These 4-D spaces are potentiated in contrast to null space because they represent positions and field organization where energy can be held, although the space currently contain no energy. The space around the centerpoint is 4-D which (1) minimally interacts with other 4-D fields, (2) is self-referencing within the atom and therefore does not appear to be disrupted and (3) represents 4-D energy transfer which is not visible conventionally.

Each direction/dimension within the atom or particle has differing levels of strength and these are constantly changing and equilibrating within the atom. These varying levels provide insight about field structure and bonding even though the relative levels of strength are idealized and considered equal within the described Model.

The neutrino—The Model defines the six-dimensional singular centerpoint as a neutrino. The neutrino, a six-dimension structure, passes effortlessly through space occupied by 6/4 seiches and rarely creates a scattering event except when it hits another 6/6 neutrino centerpoint. A neutrino has neither trapped energy flow nor is self-referencing and in comparison to 6/4 spaces, a 6/6 neutrino is “hard” (like a bullet through aero gel). Neutrinos are the seminal structures associated with the formation of particles such as quarks, neutrons, electrons and their respective counterparts. While particles can form from scratch, from the neutrino outward, particles also form as mirror particles, without requiring prior formation of substructure particles.

Neutrinos have been shown to have triple oscillation but no electromagnetic charge, passing through most matter easily and conforming to the limits of light speed. Most neutrinos have three oscillations as the result of three pairs of opposing waves that provide energy traps within the 6/6 structures. The neutrino traps the same energy as photons within a 6-D point.

6/4-D Axial Triplets, Ten Cones and Five Radical Axes

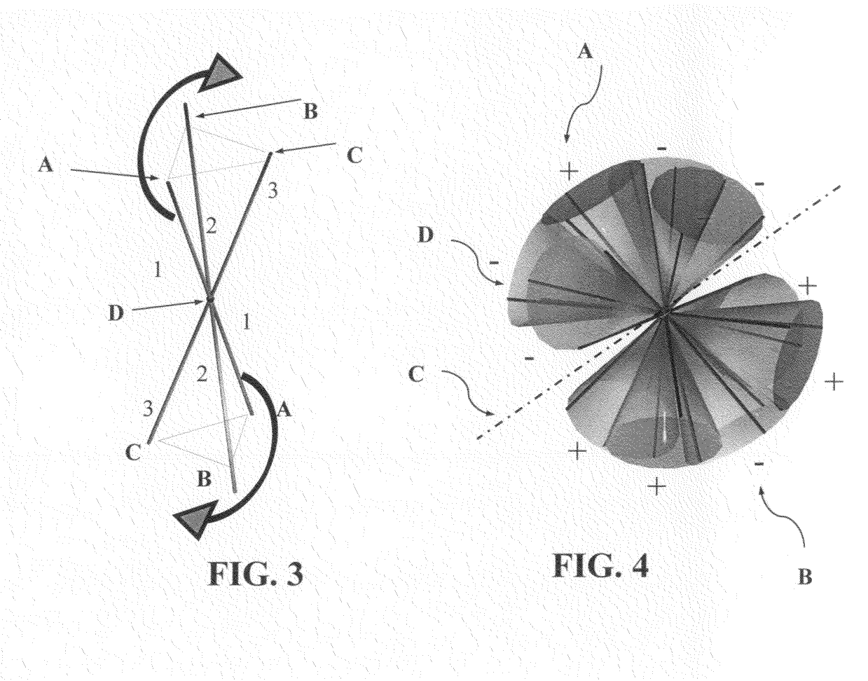

The Model defines all particle formation is organized within five sets of three 6/4 axes within the atom, defined in the Model as “axial triplets” as shown in FIG. 3. Within the atom there are five sets of triplets—that converge through the centerpoint and therefore create ten “cone” sets—of 6/4 triplets with the centerpoint at the very tip of each cone. Each cone shares the single 6-D centerpoint as shown in FIG. 4. Since each axial triplet is part of the centerpoint, the scattering forces within the atom are measured through the centerpoint. The 6/4 axial triplet space is modeled as conically, axially and locally symmetric and self-referencing. All atomic symmetry is describable using the axial triplet structure. The triplet also defines spin-up and spin-down related to the electron.

The axial triplet structure provides further lattice regularization for the formation of particles, reinforcing lattice spacing that tends to zero at the centerpoint. The tendency for pairing within the atom is the result of axial triplet alignment.

The triplet axis set naturally incorporates five dimensions for each triplet cone (6 dimension particles form rarely only when 6/4 axes are aligned perfectly). A cone is a description of the axial triplet in complex motion. Particles formed within the triplet cone therefore are also 5-dimensional. The crossover or inversion point of the axial triplet defines a unique relationship between particles formed on either side; that is, while the helicoid sequence remains the same. Particles mirror each other and are self-referencing across the centerpoint. Conversely, each independent direction is involved in only 4 of the five triplet sets (except in the rare case of a 6-D particle resulting from perfect six-direction initial alignment).

The Axial Model's 6/4 structures of axial triplets are also consistent with the most popular theories on dimensions. Single dimensions describe the transfer of energy using one direction. Two dimensions describe a hypertube. Four-dimensions describe energy transfer. Five and ten dimensions describe triplet structures and associated forces of particles and gravity. Six dimensions converge to yield what we see as conventional 3-D matter and incorporate the total energy and lattice structure of the atom. Time is not needed to create space rather it is used to describe events. The dimension hierarchy of the Axial Model includes:

TABLE 3
Axial Model Dimensional Structure

| | |
|---|---|
| Conventional view/Mass | 3-D |
| Occupiable space | 4-D (equivalent to three + time) |
| Force transfer | 1-D/2-D (strings)/4-D (3 + time) |
| Particle structure | 5-D/rarely 6-D |
| Helicoid and radical axes | 5-D/rarely 6-D |
| Charge | 5-D based on particle fields |
| Gravity | 5-D (triplets) or 10-D (cone pairs) |
| Atom | 6-D |
| Neutrino centerpoint | 6-D |
| Particle cone pairs - gravity | 10-D |
| Five sets of 5-D triplets plus time | 26-D |

Because the atom has five sets of triplets, it naturally has a conceptual equator that further organizes the atom on either side of the centerpoint (FIG. 4). This equator can be used to define 6/4 axis rotation/interaction and the sequential orientation of particle spin and Hund's rule. The equator is the plane associated with separating the spin up and axially opposite spin down triplets.

Another point about the axial triplet that becomes clearer once modeled is that there are two types of rotation involved in atoms. First, conventional 3-D rotation is where the entire molecule spins like a baseball. The second type of rotation is where the cones themselves have rotation as each of the directions sweeps through 6/4 space. This rotation of directions/dimensions is fundamental to excitation states and bonding and this complex movement results in “gearing” within the atom, as all of the axes move independently, yet, are self-referencing. Even if two or three of the 6/4-D axes are held in place through bonding or electromagnetic alignment, the 6/4-D nature of the atom allows for the third axis of the cone to continue to rotate in a complex manner within the bond.

The Background of Space

If one assumes that the background of space is full of energy but is not matter, then a dimension can be considered as a real wave. The Axial Model assumes that the background of space is materially empty; that is, it is not made of matter. The background represents a material void filled with non-material waves of potential energy described in the Model as hyperplane waves. These hyperplanes or wave sets of potential energy have the character of direction, scale and power as regularizing factors.

The background has three levels of disturbance generated by the atom: (1) the formation of the electromagnetic field based on 6 independent directions emanating from the centerpoint, (2) the flow of trapped energy described by spindle torus particle geometry and defined as gravity and (3) the release and absorption of photons.

Hyperplane waves have no size limits; they are infinitely small and infinitely large. Hyperplane waves interact on fractal scales, some scales are related to the formation of matter associated with particle fields and larger scales are related to organizing the cosmos. Smaller scales also exist. Only six directions (dimensions) of hyperplane waves of the same scale are required to create mass, although infinitely more hyperplane directions, scales and power levels exist in the background of space. Mass is generated and organized locally about the single 6-D point at which the six directions of hyperwaves related to the scale of mass, otherwise known as gravity waves, converge and trap photon energy.

The background of space is not predetermined and does not exist in whole numbers. The hyperplane universe is infinitely dimensioned with an infinite number of hyperplane scales and directions. The potential energy of the universe is not uniformly dispersed. Hyperplane is the energy that is always conserved without constants. Waves of hyperplane pass through all particle matter effortlessly. Hyperplane waves are not visible, although they ultimately organize what is seen.

The Interaction of Hyperplane Waves

Hyperplane waves are background waves that interact to form constructive interference in crests and troughs. Hyperplanes interact with each other when they are (1) in phase, (2) aligned as hypertubes traveling toward or away from each other and (3) when relative power is matched. A tighter/smaller interaction is stronger than a diffuse wave interaction. This can be seen when a photon or particle passed through two polarizing slits to create an interference patter of measurable scale and frequency.

Hyperplane waves interact when the “power” levels are similar. Power is a complex value representing frequency, amplitude and quality of interaction (angle and duration). The Model includes that pairs of opposite hyperplane waves converge to form potentiated space represented by hypertubes. These hypertubes conceptually resemble closed string loops through time where a cylinder is formed. However, unlike mathematical strings, the Model includes that these intersections represent actual determinable, self-referencing positions within the metric. The hypertubes form with crests, troughs and null space that define lattice spacing.

Hyperplane interactions to form hypertubes are the first material organization of matter. While these tubes cannot confine energy to a single point, they do provide the organization of energy as evidenced by polarized interaction of photons and particles to form interference patterns in double slit experiments. These tubes are six-choose-two sets of opposed hyperwaves.

When two hypertubes cross, they create a 4-D temporary space that can hold energy. The formation of occupiable space is based on the spontaneous and sequential generation of four-dimension points within the six-dimension lattice. This four-dimension point is the smallest occupiable space for energy and includes the formation of sets of four dimensions as they align axially and converge through the centerpoint. There are 15 sets of four-dimension spaces within this six-dimension lattice structure.

Hyperplane waves are defined by the Model as waves formed by disturbances in the background. Hyperplane waves are generated on two scales: (1) locally by mass as the sequence of trapped 4-D energy flows through the particles and (2) on infinite scales traveling throughout the universe.

Mass hyperplane waves (waves associated with mass generation) are disturbances in the background associated with particle generated gravity waves, centerpoint generated electromagnetic field and photons. On one level, the “pulse” of gravity is generated by cyclic 5-D trapped energy flow within the completion paths that create particles. On a second level, the electromagnetic force is the result of the organization and potentiation of the local atomic space generated by the interaction of the six directional waves to create 4-D spaces occupiable by energy. Finally, the photons create their unique disturbance.

Background hyperplane waves are not limited by the speed of light because their frequency (scale) can be larger or smaller than required for mass formation. However, gravity waves associated with matter travel only at the speed of light because the path of trapped energy in the particle generates them. Background hyperplane waves can move faster or slower than light depending on the relative scale to the Mass hyperplane waves. Waves larger than the scale for mass can be faster (accounting for stellar mass formation further away than predicted using light speed) and smaller scales are slower.

Background hyperplane waves and interactions help to organize the cosmos. Background hyperplane waves may be one source of the missing “dark matter” gravitational/organizational force measured in the universe.

The local formation of background disturbance suitable to the spontaneous formation of additional matter is an important part of the reason why new mass forms near existing mass. The likelihood that similar power background waves and photons to fill seiches and form neutrino centerpoints are more prevalent where particles already exist and the intersection of photons and electromagnetic fields can serve as new centerpoints.

Four-Dimensional Energy Transfer

The interrelated fractal Mandelbrot and Julia Z Power math sets can be used to describe the creation of 4-D occupiable spaces without the need for time as a dimensional variable as utilized in current space-time theories. The Julia fractal set represents the math for the smallest occupiable 4-D “space” associated with matter, a seiche, and is an essential regularizer of matter. In accordance with the Model, potentially occupiable seiches form and disappear sequentially. Depending on the sequence, energy can be classically transferred from one seiche to the next. Spaces form and disappear spontaneously unless occupied by photon energy, where they can be temporarily sustained.

There are several factors that are important about the math used to represent the transfer of energy. The Julia fractal provides a mathematical model for energy held within the seiche or transferring to another seiche. Connection describes the energy held within the seiche. Disconnection describes energy transferring out of the seiche. Connection and disconnection are also features of string theory “pants”. Finally, the Julia drawings show that only one of the four-dimensional variables needs to change to cause the fractal to begin transferring energy. Any periodicity to the variables creates movement along a complex path. The Julia Z Power fractal is Zₙ₊₁=Zₙ²+K where K is a fixed complex number and where Zₙ does not equal infinity.

LlJ={cεC|lim(n→∞)Zₙ≠∞ Where: Z₀=c (2)

Zₙ₊₁(Zₙ)²+K

The Julia set represents a 4-D space, a point, that spontaneously appears, fills in with energy and then either: (a) the trapped energy transfers forward to the next seiche through changes in the mathematical parameters, (b) the energy is held temporarily and then dissipates into the background or (c) the energy retreats back down to the centerpoint. The Julia set also provides clearer descriptions for tighter, “curled” or iterative space and reservoirs of potential energy.

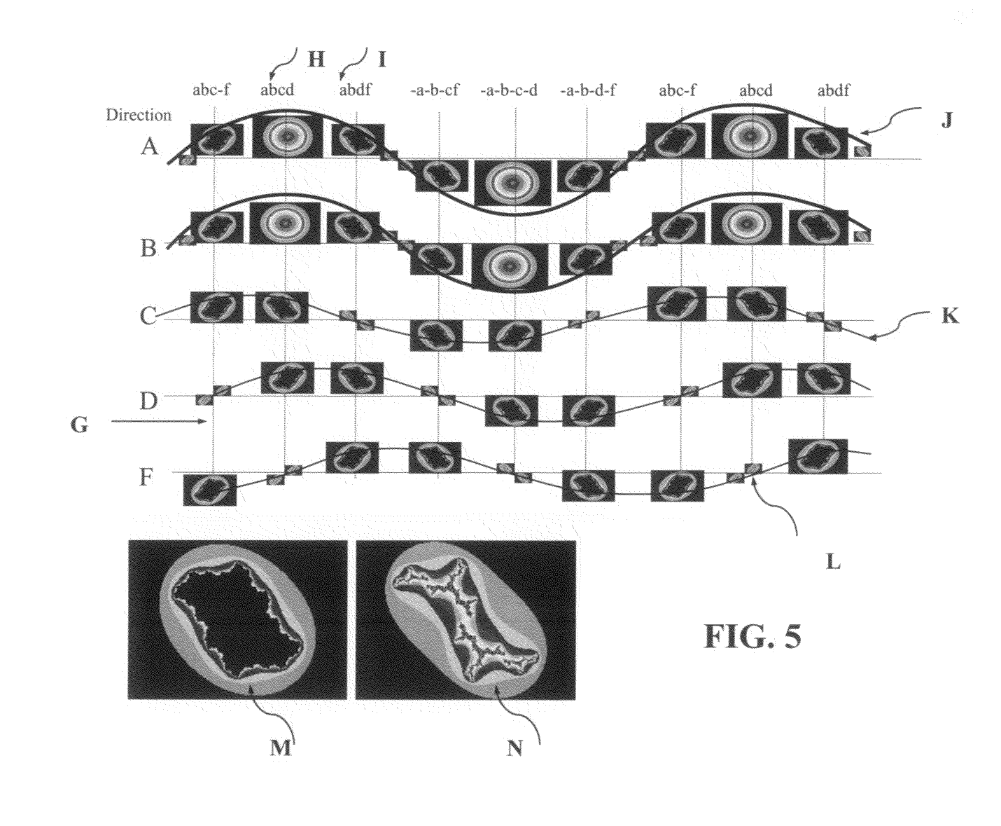

The Julia fractal also illustrates that each of the three completion paths within the particle is made up of five-dimensional sets of periodicity (FIG. 5). Within a particle or photon, a single wave direction occupies two or three full seiches. In other words, a photon fully occupies at least two points and sometimes three points in a single completion path at any given time. Further, the Model includes involvement of an advanced point where the fractal is reconnecting about one-third of a wave ahead of the first seiche (real variables 0.665, 0). The disconnection also occurs as one-third of a trailing wave of similar magnitude, although it is considered a real wave without reverse-time implications.

Flow Through High-density Lattice Points and Circle Completion Paths

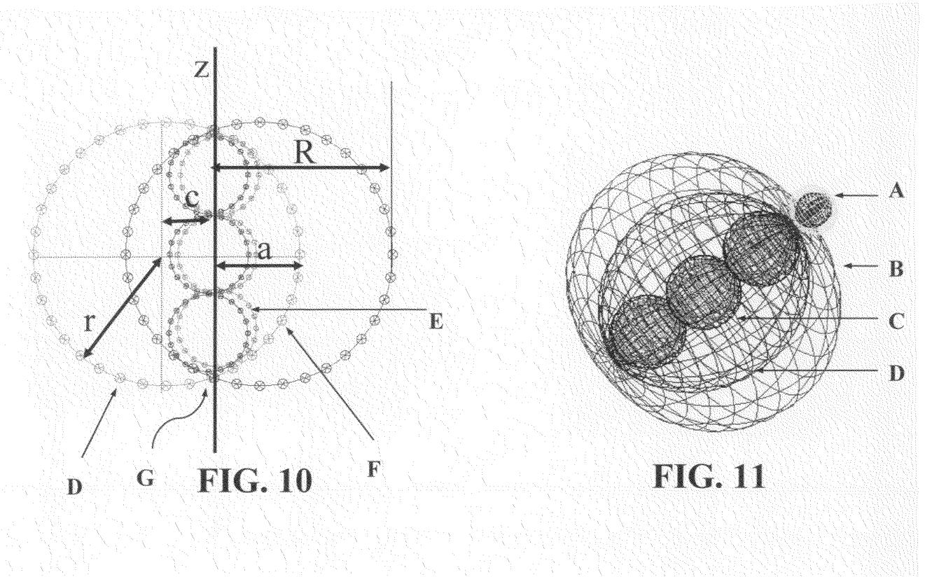

High-density circle sets and lattice density provide regulation for discrete levels of mass that are uniformly consistent between all particles on discrete scales. For trapped energy to flow within a triplet lattice system, ordered completion sets must be constructed. It was discovered that in order to flow effortlessly, the energy transfer had to occur between sets of high-density lattice points in a circular connected path lining up seiches using three separate 6/4 axial sets. The complex rotation and sweeping of dimensions/directions about the centerpoint of the axis provides proper timing and periodicity to allow additional completion sets to form, thereby creating a spindle torus. Axis sweeping is set in motion within the atom initially by the not-so-perfect alignment of the six directions intersecting at the centerpoint causing the formation of off-center oscillations within the neutrino. This motion affects the sequential alignment of seiches on both sides of the centerpoint. This atomic character is self-referencing, causing changes on both sides of the centerpoint. The high-density lattice sets represent the radius of the spindle torus tube FIG. 10 “r” and account for discrete particle sizes.

For energy in one crest of a 6/4 seiche to transfer to another seiche in the same 4-D lattice set, the energy has to transfer through two other 6/4 axial lattice sets within the axial triplet. This is why the three completion paths within a triplet are unified, allowing “5-D particles” created from three sets of 6/4 lattice sets. Each path contains 6/4 positions from each of the sets in order.

These “5-D” sets are built with three sets of 6/4-D high-density lattice paths and represented visually as a 2-D circle. Using the Axial Model's sweeping direction/dimension, whether the points are equidistant does not matter as long as the distance between seiches remains below a maximal distance between seiches that energy can be transferred across. This distance may be related to Planck length. If energy is not transferred forward, the energy can be held temporarily in the seiche to wait for a suitable seiche to form. If the forward seiche is not available, the completion path backwards will be taken or else the energy is lost to the background. This structure allows for a smooth energy transfer rather than jumps from point to point as required by the Standard Model.

Within every particle with mass, there are paths of trapped energy flowing through the 4-D metric that follow specific 6/4-D paths associated with high-density lattice circle sets. These sets define the specific hierarchal masses of particles, including the proton on down to single dimension string force levels, 4.69E-21 relative to the proton.

Mass and completion paths—Mass is defined by the Model as particles with 4-D energy flowing within three simple circular completion paths following a spindle torus geometry. The Model defines the single path of energy transfer as a completion path. A completion set includes the three completion paths that make up particles.

The completion path can involve as few seiches as three upward and three downward (sub-pentaelectron). This structure represents the smallest particle with trapped flow and therefore mass. Energy transfers from seiche-to-seiche within the local system at the speed of light. In most higher mass matter, the transfer from seiche-to-seiche continues at light speed but the availability of the next seiche is limited by the rotation of the direction making the transfers appear to be moving slower.

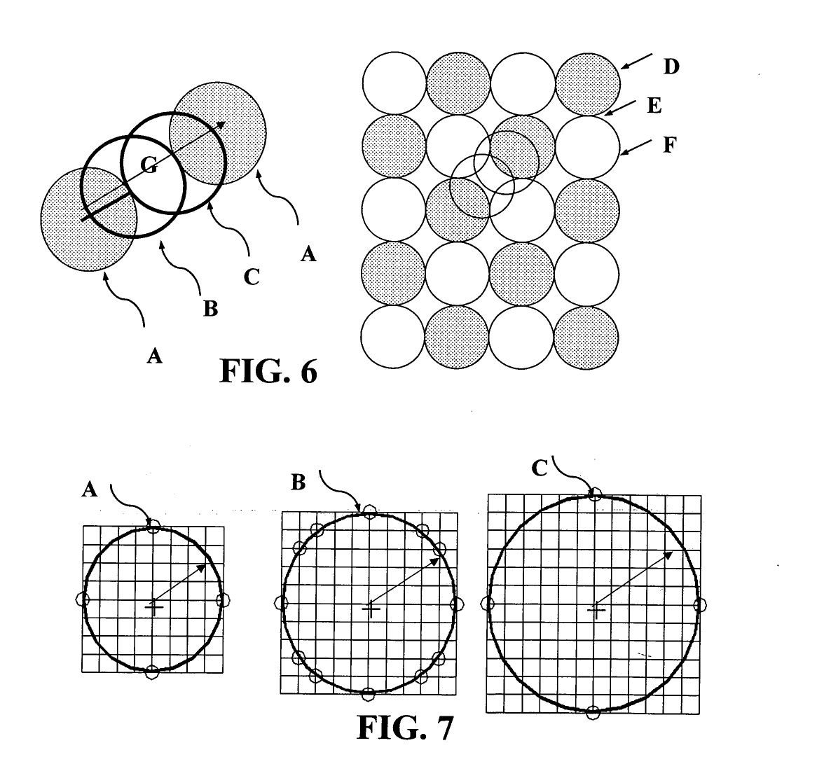

Each completion path involves transfer through two additional lattice sets in order to return to the starting centerpoint. In other words, to transfer from seiche crest to seiche crest within lattice ABCD, energy has to go through two other lattices ABCD and ABDF within the triplet (FIG. 6).

The number of occupiable seiches on a completion path is based on specific high-density solutions to circle lattice equations. The path of the trapped energy follows these circular paths on each of three axes in the axial triplet (in a 3-D view). The circle having n lattice points, radius r, center (0,0) is calculated in the following manner:
A. Prime factorization. Every positive integer n>1 has a unique factorization in the form

n=2^{a}(Π_{p=1 mod 4}p^{b})(Π_{p=3 mod 4}q^{c}) (3)

where the p's and q's are prime numbers. Unless specified otherwise, in what follows, p always denotes a prime number of the form 4k=1 and q always is a prime number of the form 4k+3. We shall denote

P=Πₚp^{b} (4)
B. Expression of an integer as sums of 2 and three squares. For every positive integer n, we write

a. r₂(n) as the number of pairs of integers (x, y) satisfying x²+y²=n,

b. r₃(n) as the number of triples of integers (x, y, z) satisfying x²+y²+z²=n.

These functions, though known, are very tedious to calculate for lattice points on circles of integer radius, however, the expressions are reasonably simple.
C. Lattice point on circles. The number of lattice points on the circle radius n, center (0, 0), is

r₂(n²)=4Πₚ(2b+1) (5)

Remark: There is another useful expression,

r₂(n²)=4(d₁(n²)−d₃(n²)), (6)

where j=1, 3, dⱼ, (n²) is the number of divisors of n² of the form 4k+j. As shown on Table 4, the number of lattice points for a given radius are calculated. At radius of five, twelve lattice points are on the circle. At radius of 25 there are 20 points on the circle. At radius of 65, the first showing of 36 lattice points appear. Similar counts of lattice points appear at many scales.

TABLE 4
Number of Lattice Points on Circles of Radius n < 190.
(Radius n = sum of row and column numbers)

| | 1 | 2 | 3 | 4 | 5 | 6 | 7 | 8 | 9 | 10 |
|---|---|---|---|---|---|---|---|---|---|---|
| 0 | 4 | 4 | 4 | 4 | 12 | 4 | 4 | 4 | 4 | 12 |
| 10 | 4 | 4 | 12 | 4 | 12 | 4 | 12 | 4 | 4 | 12 |
| 20 | 4 | 4 | 4 | 4 | 20 | 12 | 4 | 4 | 12 | 12 |
| 30 | 4 | 4 | 4 | 12 | 12 | 4 | 12 | 4 | 12 | 12 |
| 40 | 12 | 4 | 4 | 4 | 12 | 4 | 4 | 4 | 4 | 20 |
| 50 | 12 | 12 | 12 | 4 | 12 | 4 | 4 | 12 | 4 | 12 |
| 60 | 12 | 4 | 4 | 4 | 36 | 4 | 4 | 12 | 4 | 12 |
| 70 | 4 | 4 | 12 | 12 | 20 | 4 | 4 | 12 | 4 | 12 |
| 80 | 4 | 12 | 4 | 4 | 36 | 4 | 12 | 4 | 12 | 12 |
| 90 | 12 | 4 | 4 | 4 | 12 | 4 | 12 | 4 | 4 | 20 |
| 100 | 12 | 12 | 4 | 12 | 12 | 12 | 4 | 4 | 12 | 12 |
| 110 | 12 | 4 | 12 | 4 | 12 | 12 | 12 | 4 | 12 | 12 |
| 120 | 4 | 12 | 12 | 4 | 28 | 4 | 4 | 4 | 4 | 36 |
| 130 | 4 | 4 | 4 | 4 | 12 | 12 | 12 | 4 | 4 | 12 |
| 140 | 4 | 4 | 12 | 4 | 36 | 12 | 4 | 12 | 12 | 20 |
| 150 | 4 | 4 | 12 | 4 | 12 | 12 | 12 | 4 | 12 | 12 |
| 160 | 4 | 4 | 4 | 12 | 12 | 4 | 4 | 4 | 20 | 36 |
| 170 | 4 | 4 | 12 | 12 | 20 | 4 | 4 | 12 | 4 | 12 |
| 180 | 12 | 12 | 12 | 4 | 36 | 4 | 12 | 4 | 4 | 12 |

Completion paths of highest density occur in discrete size levels, involving radial multiples of five and point values divisible by four. As shown in FIG. 7, high-density lattice sets occur naturally and result from the selected radius. In a circle of radius five, 12 lattice nodes/points lie on the circle (a useful high-density lattice set), while using a radius of four or six, only four lattice nodes are intersected.

Shown on Table 5 are the numbers of lattice points (x, y) on the circumference of a circle of radius n with center at (0,0). Each radius represents important lattice seiche counts and represent building sets of the high-density among all choices. There have been no skipped values. Some of the particles revealed by the Axial Model are newly discovered. Additional scale particles are available at many levels of energy and can be created in the laboratory, however, the sets associated with conventional matter and low energy represent the base sets occurring naturally.

TABLE 5
High-Density Lattice Sets

| | | # Lattice Points |
|---|---|---|
| | | on High-Density |
| Particle | Radius | Circles |
| Seiche | 0.5 | 1 |
| Sub-pentaelectron | 1 | 4 |
| Pentaelectron | 5 | 12 |
| Sub-Pentaquark | 13 | 12 |
| Electron Quark | 25 | 20 |
| Pentaquark | 65 | 36 |
| Electron | 85 | 36 |
| Quark | 325 | 60 |
| Proton | 1105 | 108 |

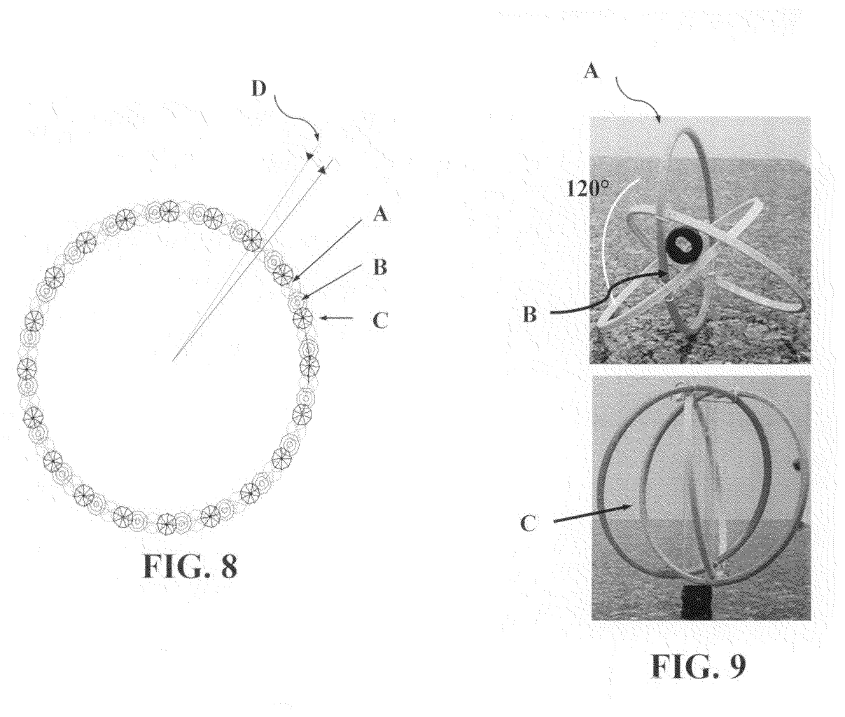

A full completion path uses seiches from each of the triplet axes and therefore the path is five-dimensional. Each path contains three sets of 6/4 lattice points and each particle is made up of three completion paths (FIG. 8). For the purposes of representing the Model and the relative scale of particles, the completion path is shown with a single 6/4 set. The unified triplet produces three paths that have the same sequence within the particle, but they have slightly different positions relative to the centerpoint or lemon density. Photons emitted from any of these paths within the particle have the same frequency.

Finally, a single direction rotating to a higher or lower energy state can be still be completed with more than one solution, shifted slightly and still complete (contributing to the Lamb shift and hyperfine splitting of spectra). The distance that energy can be successfully transferred is maximally limited. The completion path is only shown in 2-D. In 4-D, the path crosses near the centerpoint twice in a twisted figure eight, un-renderable and somewhat misleading in 3-D. Therefore, herein, the completion path will be shown as a 2-D circle. Further, when calculating the relative energy of particles, the seiche count is reduced to a single variable. Since each seiche is made up of four dimensions within a periodic structure, the relative energy described by a seiche is described as (ABCD)² or (n⁴)². For the model this is simplified to a single variable.

The Spindle Torus Particle and Resident Energy

To be considered mass, a particle must contain three completion paths. On an ordered basis, the completion for each particle involves three separate trapped energy paths, organized within the axial triplet (FIG. 9), ultimately generating a spindle torus in 6/4-D about the radical axis. A triplet sequence includes starting the sequence from three different positions around the centerpoint.

The three completion paths ultimately develop into a complex spindle torus with radii matching the high-density lattice sets. The spindle torus, based on high-density seiche energy transfer, yields a very unique Model in that the particle appears to be a uniform shell spinning about a definite axis with almost all of the mass held on the surface. In all cases, the completion path follows a sequential seiche path (right or left) aligned with the crests and troughs associated with the local sweeping 6/4 fields and sequential formation of seiches. Each of the three paths in the completion set is offset averagely about 120°.

The path for all particles starting from the centerpoint is initially upward (away from the neutrino centerpoint), then over the outside and downward, back to the centerpoint. This is because the centerpoint releases energy outward along its 6/4 axes and organizes smaller particles to be nested within bigger particles that form. As smaller particle scales complete, larger ones form over/around the sub particle set outside using larger high-density completion paths. Each of the three completion paths builds around a 6/4 axis in the triplet, overlapping around the radical axis, naturally creating a spindle torus. The radical axis is the centerline, equidistant from each of the three 6/4 axes, in the triplet. The radical axis represents the straight centerline of the torus and the centerline of axial alignment of each triplet particle.

The three paths sequentially transfer energy from seiche to seiche at the speed of light as new seiches become available. As the paths develop, a very distinct spindle torus “apple” and “lemon” character appears. The shape of the lemon is a Reuleaux shape (three-sided football). The multiple flow paths within the torus create intersecting planes within the torus, intersecting at the radical axis of the torus and triplet.

The atom's energy is never balanced; rather, there is a constant ebb and transfer of energy within particles in the atom. Within the atom, at any given moment, there are seiches that are unfilled/incomplete or “weak” as well as seiches that are full or otherwise “strong.” Photon absorption is an indication of new photons transferring energy to an incomplete system. Equilibration continually occurs between particles through shared directions, seiches, cones and through the centerpoint.

The structure for complex spindle tori is shown in FIG. 10 and Table 6, where R is the radius of the outside of the torus tube from the centerline, r is the radius of the inside of the tube, c is the distance from the centerline of the torus to the center of the tube and where h=(r²−c²)^{1/2}. Shown are two of the three completion paths associated with an electron and electron quarks.

TABLE 6
Complex Spindle Torus Volumes (7)

| | |
|---|---|
| 3-D volume | W₃ = 2πh(2r² + c²)/3 + 2πcr²(π − arcsine(h/r)) |
| 4-D volume | W₄ = (π²/6)(3r − c)(r + c)³ |
| 5-D volume | W₅ = (πr²/2)(W₃) − (2π²h⁵)/15 |
| 6-D volume | W₆ = (2πr²/5)(W₄) − (π³h⁶)/30 |

To determine the scale of particles, the circle radius “r” used for each of the spindle tori is taken from radii for high-density lattice sets. These radii exactly match the scale of sub-atomic particle sizes. Combining the three rotating planes generated by the sequentially rotating completion paths, FIG. 11 shows the structure for the hydrogen atom including quarks based on many cycles of the completion paths through time.

The Radical Helicoid

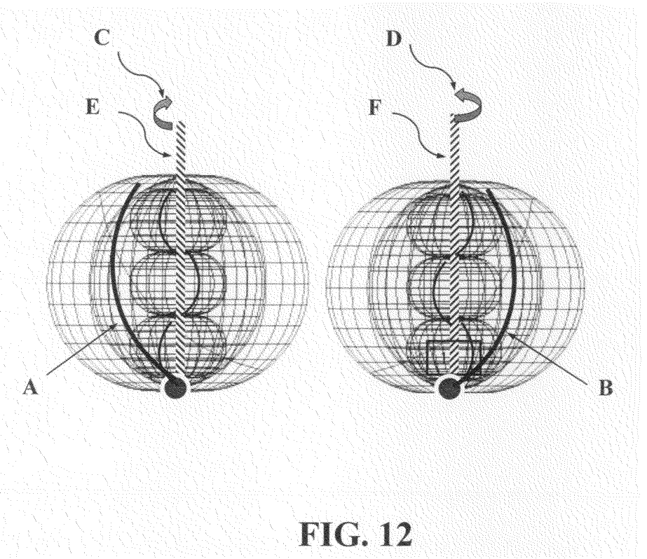

The radical helicoid organizes electromagnetic fields, charge, gravity and formation of photons. The three rotating planes of the particle's completion sets create a helicoid through the radical axis of the Vesica Piscis (torus lemon) along the radical axis center of the spindle torus (FIG. 12) and align with the center of the axial triplet. The rotation and character of the radical helicoid are determined by the order of the three completion paths and the sequence, excitement levels and relative radius of the particle. As each path circulates in sequence, the auger of the helicoid determines handedness for the particle.

For a triplet to transfer energy and be confined within the torus geometry, the lattice points for a given completion set straddle the centerline of the torus, sharing seiches with the other two lattice sets in the triplet in each of the three paths of the completion set. For example, the first lattice point of the proton is at 1.6666° on either side of the radical helicoid within a radius lattice set of 108 seiches/360° (3.3333° between neutron and proton lattice points).

The helicoid's coherence is affected by the degree of overlap of the three “planar” completion paths of the spindle torus. Chemical bonding is substantially controlled by the character of the electromagnetic field and respective helicoid axes to be bound (tightness, alignment, twist). Importantly the coherence between atoms of the individual directions, 6/2 hypertubes and 6/4 lattice sets is the determiner of the angles and strength of bonds. Spectral hole burning and zero phonon structures are a result of the organizing effects of the helicoid axes.

Axial and Mirror Symmetry

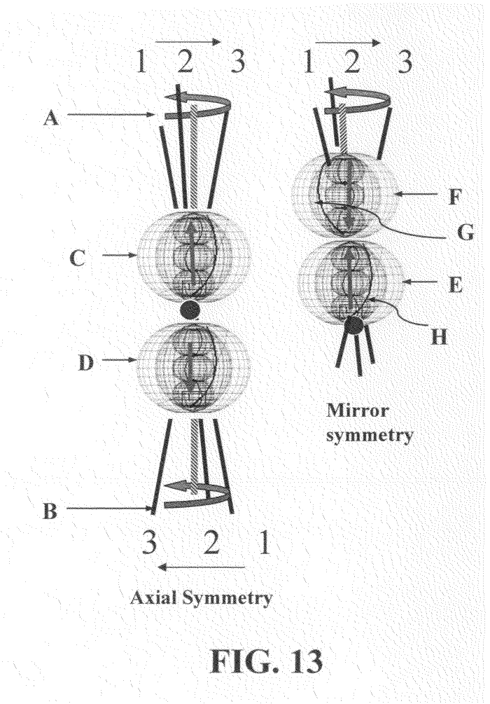

The axial triplet and 6/4 structure account for atomic symmetry as shown in FIG. 13. The Model defines two types of symmetry: mirror and axial. While this is well understood in chemistry, it is a novel construct offered by the Axial Model and related directly to the organizational structure of the 6/4 metric and triplets.

Mirror symmetry is symmetry on the same side of the axis. Protons/neutrons and axial triplet groups of quarks are examples of particles exhibiting mirror symmetry. Mirror particles share seiches.

Particles easily form through mirror symmetry as the result of sequential transfer of energy through shared seiches, substantially in sequence with each other as a result of individual direction sweeping. Mirror flow is the result of minor changes in the Julia variables such that at the right time, the flow path is changed for one of the four dimensions to follow an alternate mirror sequence, which eventually represents a new particle. Maintaining all other variables causes the path to come back to itself. These changes can be the result of increased spin, additional photons or other parameters that affect the timing of the trapped flow. It is important to note that the radical center flow of contiguous mirror particles is in opposite directions.

Axial symmetry is exhibited across the centerpoint because the axial triplet flow is inverted on the other side of the centerpoint. Axial symmetric particles share the centerpoint. Neutron/neutron pairs are axially symmetric across the centerpoint. The axes within the triplet cross over at the centerpoint, underpinning the framework for equal and opposite symmetry, handedness, entanglement and field effects. Axial symmetry is shared through the centerpoint. When the axes invert, so does the helicoid sequence, causing the auger direction to be opposite spin, yet the axis-to-axis sequence remains the same. Axial symmetric particles have opposite handedness as the result of axial sequence changes across the centerpoint.

Matter tends to form within doublet, triplet and quintuplet particle sub-structures. There are numerous relationships between position of the different particles and the resonant properties they develop. The character of each sub-particle is based on the relative seiche and trough positions and flow involved: mirror or axial.

Doublets often share the centerpoint. Triplets form when two particles at the end of the triplet have resonant mirror flow and the direction of the flow path of the first and third particles causes an additional proton/neutron “cone level” or spindle torus path over the outside. Particles can form from “scratch” building from the neutrino up to a proton by hierarchal steps, or a particle can from contiguous particles matching the scale and geometry of the original particle within the range of it electromagnetic field.

Entanglement is achieved by separating axial and mirror symmetric particles. Change the direction/character of one mirror/axial symmetric particle within the atom and the particle or photon changes on both sides of the triplet. The spin and character of the photon or particle is determined at the triplet.

Mass Gap and Shared Seiches

Mass gap and confinement are both a function of sharing contiguous seiches in their respective completion sets and between contiguous particles within a triplet. Sharing seiches also accounts for the tremendous strong nuclear force within the atom.

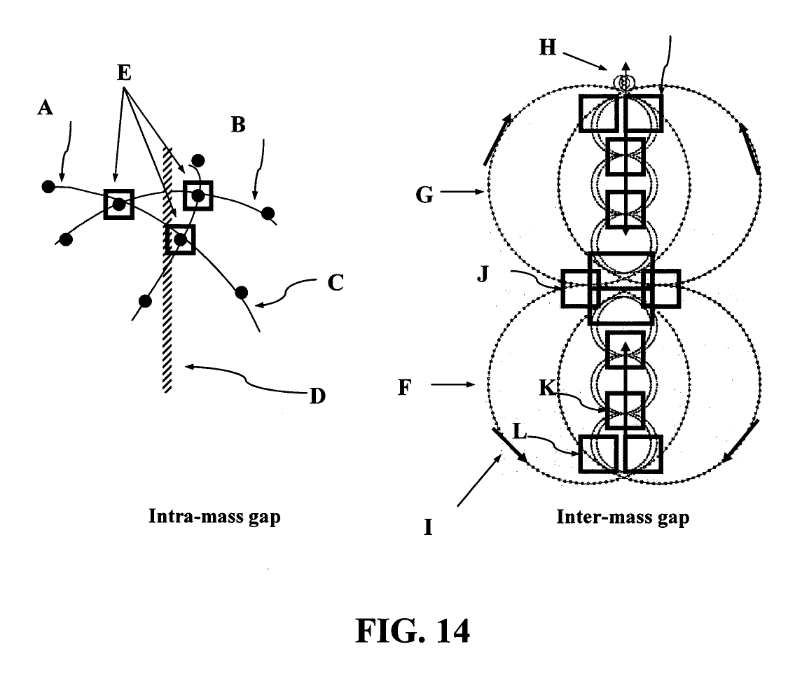

Mirror symmetry particles share seiches on the completion path (FIG. 14). Axially symmetric particles only share the centerpoint. The energy of the atom equilibrates through sharing seiche spaces. For example, of the proton's 108 seiches in one 4-D completion path, the proton shares one seiche with the neutron or 0.94% (1/106.4) of that path.

Particles and respective sub-particles also share seiches. Finally, completion paths share seiches.

The Model includes a total of 108 seiches that make up the completion sets of both the proton and neutron, at least one seiche per 6/4 axes is shared between symmetry particles, accounting for mass gap. The interlocking system of dependent flow ensures the durability of protons even without dependence on the neutrino. The intersecting completion paths at each end of the torus lemon and the doubled completion path in 4-D provide the “density” observed as the nucleus in the hydrogen atom and electron which are 5-D objects. As a result, hydrogen does not always cause scattering in Rutherford style experiments. Larger particles are all built around a neutrino.

Confinement—Major and Minor

Confinement is the structure of smaller particles trapped within the lemon of the spindle torus and is based on sharing seiches and geometry that naturally facilitates the spindle torus geometric structure. In order to have larger completion paths, completion path seiches must be directionally aligned with sub-particles to create confinement. This requires that the first and last positions of the confined sub-particles have to have the same seiche sequence and match the geometric position for a shared seiche. This dictates that major and minor confinement have sub-structures of odd numbers of sub-particles. While almost any scale particle can be made in the laboratory (numerous completion paths exist at higher energy scales and larger relative radius) using major and minor confinement, the structures cited above are the most prevalent.

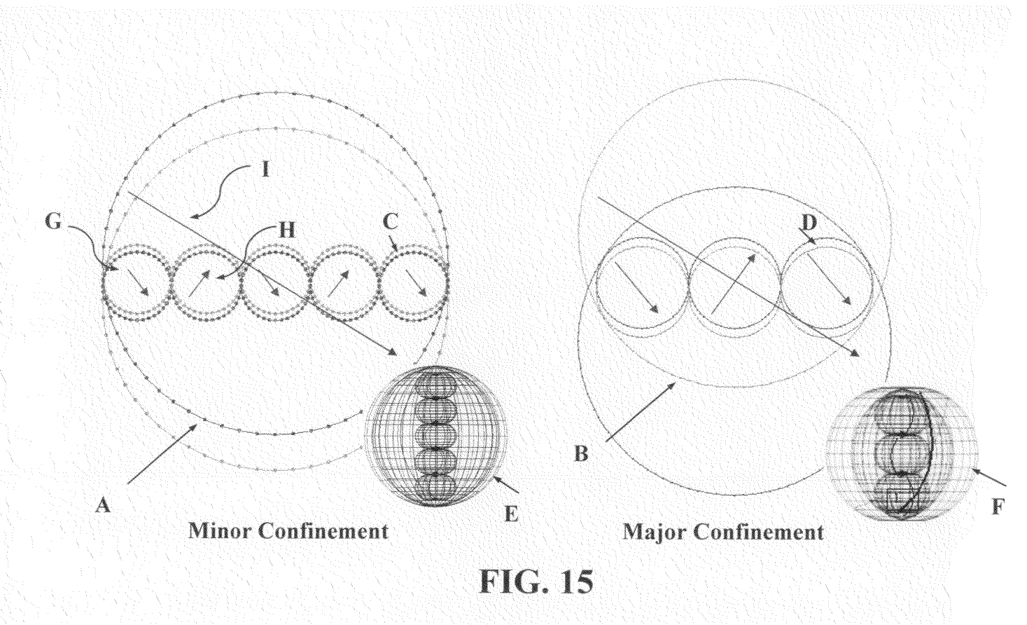

There are two classifications of confinement defined by the Axial Model: (1) major confinement, and (2) minor confinement (FIG. 15).

Major confinement—Major confinement (e.g., electrons and protons) is where three sub-particles share the inside of the torus lemon. The length of the lemon is approximately 94% of the diameter of the torus and the torus overlaps at about 65%. The percent overlap will vary depending on the character of the six-direction interaction. These are very stable particles, because the energy within the quark completion paths is held within the proton's lemon with no direct escape to the outside of the particle. The ratio of the radius of the proton to confined quarks is exactly 3.4 to 1. Quarks only appear to jump around within the proton because they are also 5-D particles objects. In fact, the Reuleaux lemon structure provides tremendous conformity to the radical axis and consequential helicoid for all particles and sub-particles.

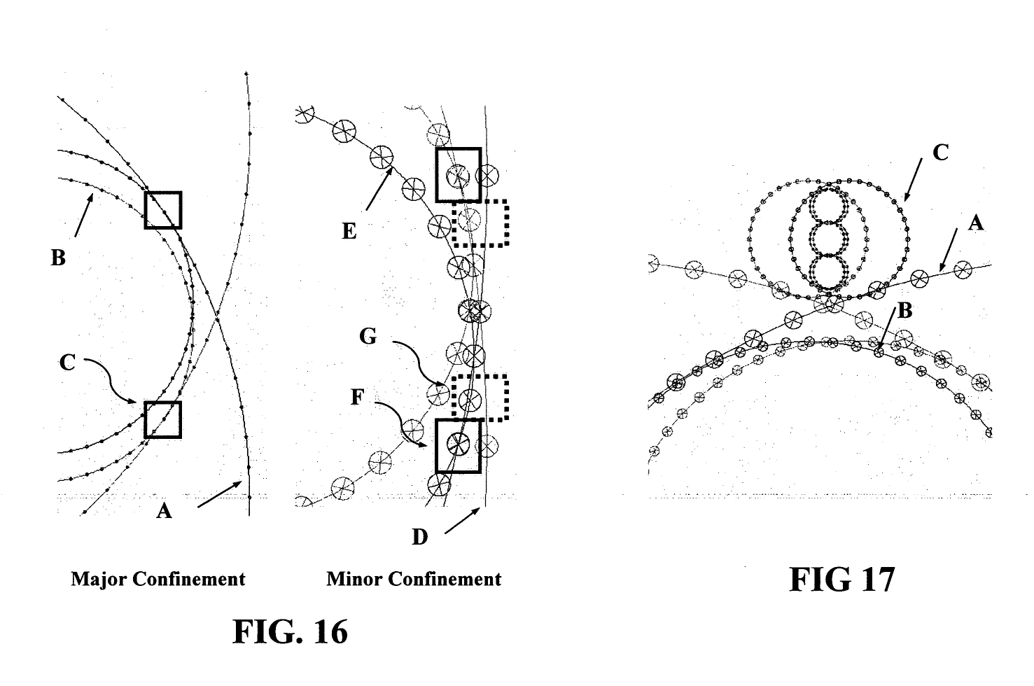

Minor confinement—Minor confinement is where five sub-particles share the lemon of a torus particle. The radius of the torus tube to the sub-particles for a minor confined particle is exactly 5:1. Pentaquarks have a lemon length of about 99.6% of the diameter of the quark torus tube, or 90% overlap. The quark confines the pentaquark at the quark's 30 seiche point, matching the geometry and sharing seiches with the first and fifth pentaquarks at their 150 seiche points, yielding a total lemon length of 99.6% or 90% overlap (FIG. 16).

What is remarkable about the pentaquark, however, is that it has lattice points at 5° that are not confined by the quark structure, placing pentaquark seiches actually outside the quark, accounting for the lack of confinement and short stand-alone quark lifetime.

Electrons also share seiches with the attractive end of protons, although the shared seiches are not confining (FIG. 17). Electron positions are lost when the completion path flow of either the proton or electron is disrupted and the shared seiche positions are not available (e.g., in the case of plasma). Electrons also lose position when the excitation level of the proton or electron not longer match their respective positions.

Discrete Particle Sizes

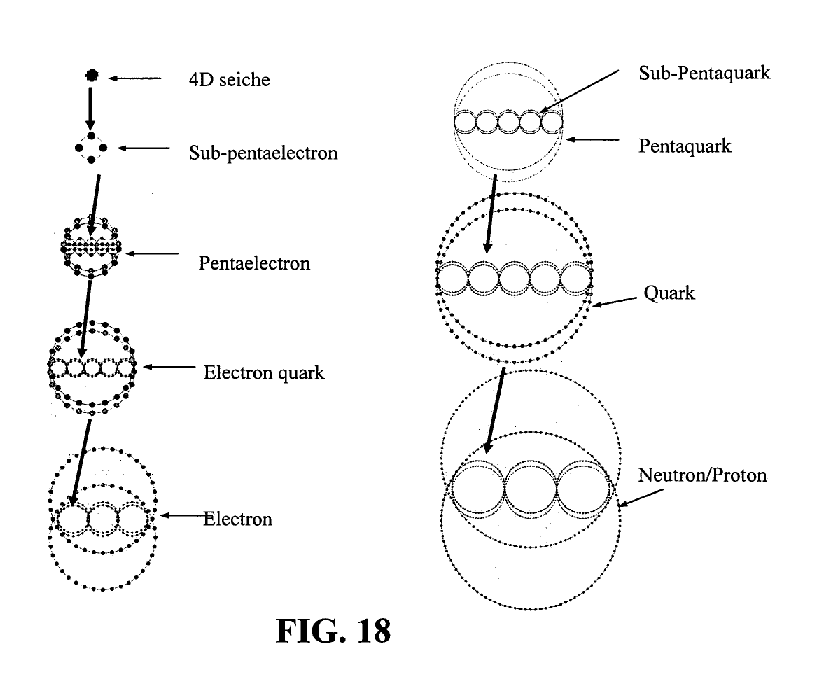

The Axial Model includes the discrete structure of particles from the individual proton down to the individual seiche. The Model reveals that protons have two substructures, a quark and a pentaquark (FIG. 18). The Model also reveals that electrons have a substructure that consists of an electron quark, a pentaelectron and a sub-pentaelectron. Mass is measured in three dimensions while the energy for the atom, in its entirety, can be expressed in six dimension thereby unifying the scale of the proton to Planck length 20 orders of magnitude smaller.

The Model is highly accurate in that it matches the scale of the electron to the proton to eight orders of magnitude. The Model calculations further reveal that mass is measured in three dimensions, as is conventionally understood. While it is recognized that there are an unlimited number of potential particles based on higher energy set particles that can be created in a laboratory, the Model focuses on particles that occur at conventional energy levels.

Surprisingly, the Axial Model reveals that the electron and proton are each major confinement particles that have triplet sub-structures and a more spindle torus structure at approximately 65% overlap or a lemon length of 94% of the torus tube diameter.

There are just two adjustments to the raw torus data that are required to calculate the relative mass of particles, (1) the relative seiche density and (2) the mass gap associated with the intersection of the completion path seiches at the ends of torus lemon.

First, the radius and seiche count of the particle torus structures reveal the particle's seiche density relative to its actual torus volume, an important part of calculating relative masses of particles. The relative 4-D seiche densities (Table 7) reveal that the smaller particles have a higher relative density (completion path seiche count/radius). This has been translated below into a relative seiche density factor, which is a function of the seiche count divided by the “r” torus radius and then normalized to the proton.

TABLE 7
Relative Seiche Density by Particle

| | | | | | Intra- |
|---|---|---|---|---|---|
| | | | | | Mass |
| | | | 3D Density | 3D | gap |
| | | | Seiche | Density | Shared |
| | | Path | count/radius | Density | seiche |
| | Radius | Seiches | Value | Vs Proton | Count |
| Proton | 1105 | 108 | 0.097738 | 1.00000 | 1.6 |
| Electron | 85 | 36 | 0.423529 | 4.333333 | 1.6 |
| Quark | 325 | 60 | 0.184615 | 1.888889 | 1.6 |
| Pentaquark | 65 | 36 | 0.553846 | 5.666667 | 1.6 |
| Electron Quark | 25 | 20 | 0.800000 | 8.185185 | 1.6 |
| Sub-pentaquark | 13 | 12 | 0.923077 | 9.444444 | 1.6 |
| Pentaelectron | 5 | 12 | 2.400000 | 24.555556 | 1.6 |
| Sub-pentaelectron | 1 | 4 | 4.000000 | 40.925926 | 1.6 |

Second, mass gap or shared seiches within the particle completion set are found at the points where the completion paths intersect at the ends of the torus lemon. Each of the three completion paths share seiche with the other two completion paths as they cross. This has the effect of reducing the measured mass of any particle by a determinable amount of 1.6 seiches. This is important because the seiche count and density are the primary determiner of 3-D mass measurements. This phenomenon can also be seen in mass gap loss associated with contiguous axial and mirror symmetric particles.

The rules for intra-mass gap are the same for most particles (except the neutron and other centerpoint-bound particles): at each end of the lemon, the path “ABCD” shares one 4-D seiche with path “ABCF” and one seiche with path “ABDF”. These are added [(4+4)/5-dimensions=8/5] and multiplied by 2 to account for both lemon ends (8/5*2=16/5). Assuming 50:50 sharing this product is divided by 2 to yield 1.60 unique seiches per completion path lost to intra-mass gap within the particle. For the proton, this means a reduction from 108 unadjusted lattice points to 106.4 adjusted lattice points. The electron drops more mass on a percentage basis, from 36 lattice points to 34.4 adjusted points relative to the proton.

Neutrons have the added 6-D centerpoint, which is the origin of the radical helicoid and fits within the radical center of the completion path intersection. This adjustment adds back a 6/5-D value to the calculation and thereby increases the path count seiches by 1.2 seiches which accounts for the larger apparent neutron mass.

The Axial Model reveals the hierarchy of mass particles from the sub-pentaelectron up to the proton. The hydrogen proton is made up of 108 lattice points with a radius of 1105 lattice points.

Table 8 shows some of the variables to determine the relative 3-D masses of these particles. To calculate the seiche density, the torus r radius is multiplied by the adjusted seiche count/radius value (less intra-mass gap); the seiche count is normalized and then simplified to match the simple seiche count by reversing the (n⁴)² power of the four-dimensional seiche.

TABLE 8
Particle Surface Density Based
On Seiche Count and Torus Radius

| | | | Intra-Mass Gap | Count/radius | (n⁴)² Power |
|---|---|---|---|---|---|
| | Torus tube | Raw | Adjusted | Scale to Proton | Mass |
| Particle | Radius | Seiche count | Seiche count | Normalized | Adjustment |
| Proton | 1105 | 108 | 106.4 | 1.00000000E+00 | 1 |
| Electron | 85 | 36 | 34.4 | 4.20300752E+00 | 1.1965890 |
| Quark | 325 | 60 | 58.4 | 1.86616541E+00 | 1.0811072 |
| Pentaquark | 65 | 36 | 34.4 | 5.49624060E+00 | 1.2373946 |
| Electron quark | 25 | 20 | 18.4 | 7.64360902E+00 | 1.2894732 |
| Sub-pentaquark | 13 | 12 | 10.4 | 8.30827068E+00 | 1.3029832 |
| Pentaelectron | 5 | 12 | 10.4 | 2.16015038E+01 | 1.4682855 |
| Sub-pentaelectron | 1 | 5 | 2.4 | 2.49248120E+01 | 1.4947859 |

Table 6 shows the adjusted volumes of the particles based on the spindle torus volume using radii of high-density lattice sets and adjusted for mass gap and seiche density. As larger completion paths and particles are formed, the smaller sub-structures dissipate.

The Axial Model also show that the scale of the proton can be related to the scale string theory and Planck length scales in 6-D down to 4.69E-21 relative to the proton (Table 9).

TABLE 9
PARTICLE HIERARCHY
Order of Magnitude, Spindle Torus Adjusted*

| | | | Est. | Adjusted | |
|---|---|---|---|---|---|
| | Lattice Points | Radius | % overlap | 3-D Volume | 6-D Volume*** |
| Seiche | 1 pt | 0.5 | 90% | 5.7743E−10** | 4.69E−21 |
| Sub-pentaelectron | 4 pt | 1 | 90% | 6.9051E−10 | 3.00E−19 |
| Pentaelectron | 12 pt | 5 | 90% | 8.4783E−08 | 4.69E−15 |
| Sub-Pentaquark | 12 pt | 13 | 90% | 1.3224E−06 | 1.45E−12 |
| Electron quark | 20 pt | 25 | 90% | 9.3073E−06 | 7.32E−11 |
| Electron | 28 pt | 85 | 65% | 5.4465E−04 | 2.07E−07 |
| Pentaquark | 36 pt | 65 | 90% | 1.5698E−04 | 2.26E−08 |
| Quark | 60 pt | 325 | 90% | 1.7144E−02 | 3.53E−04 |
| Proton | 108 pt | 1105 | 65% | 1.0000E+00 | 1.00E+00 |

*Calculated based on spindle torus structure, adjusted for relative seiche density and mass gap within the particle (less 1.6 seiches). The mass calculation is not adjusted for mass gap with contiguous particles.
**Unable to adjust for mass gap or density on a single seiche.
***Unadjusted

Mass in 3-dimensions and energy transfer in 4-dimensions capacity of a particle are not the same as evidenced by atomic excitation states and bonding energies that change field strength, photon frequencies and influence chemical or biological interactions, but do not affect the mass of an element. The Model demonstrates definitively that mass is measured in 3-D, which is a function of the number and radius of high-density seiche points, less shared seiches, within particle completion sets, and resident energy in and out of a particle is constantly changing within a 4-D to 6-D context. The constant flow of energy within the particle structure also provides the causal structure for inertial mass and gravity mass calculated as being the same value.

The Proton and Neutron

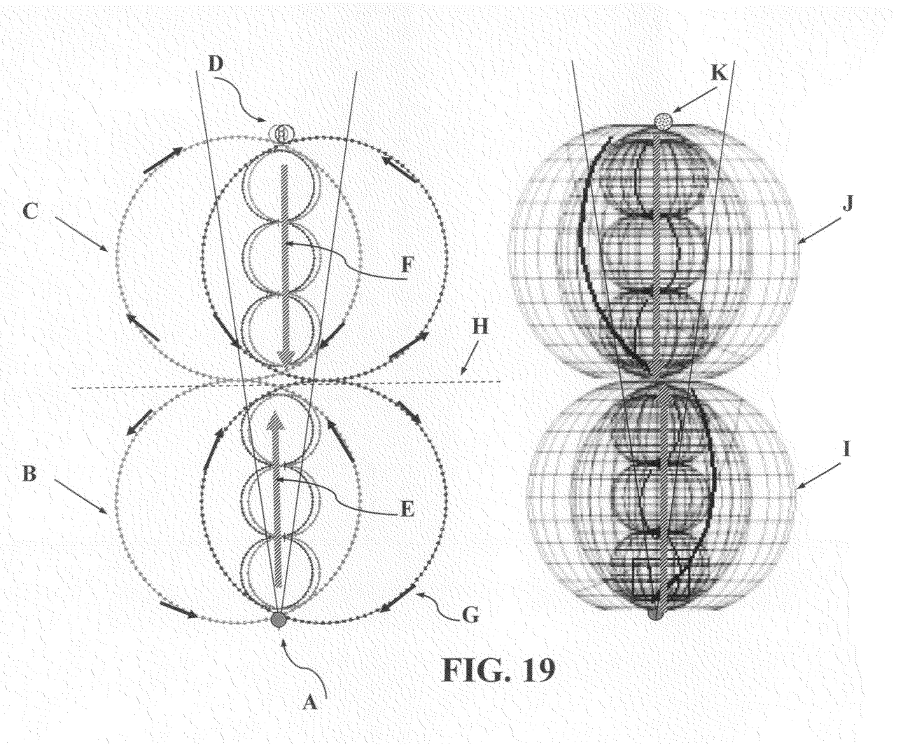

The proton and neutron have the same particle structure and radii, both having 108 seiches within the completion path of the spindle torus. The neutron has no apparent charge because the attractive side of the particle is tied to (shares) the centerpoint. The proton, on the other hand, has energy transfer through the lemon inward with the attractive part exposed, holding the electron (FIG. 19). The Model represents the axial structure of the neutron, proton and electron, where the position of the electron is determinable and is “supported” by the neutron and proton particles and respective 6/4 triplet fields.

The neutron's inside of the torus completion path flow is always outward, away from the centerpoint as a result of the build-up of larger scale completion paths over the outside of smaller sub-particles. The proton is later formed based on the mirror flow of the neutron. The neutron and proton share seiches and mirror symmetry. Opposing neutrons share the centerpoint and axial symmetry.

Protons and neutrons are formed using the same number of seiches and high-density lattice sets, 108 with a base radius of 1105. There are several differences between neutrons and protons: (1) the centerpoint acts as an extra point in the neutron completion set; (2) the neutron's attractive charge is tied to the centerpoint, effectively negating its visible charge, (3) the proton's attractive charge is tied to the electron, (4) the neutron has a tighter relative scale compared to the proton within the same lattice set and (5) the neutron draws energy from the shared centerpoint and consequently is the primary equilibrator of energy in the atom.

The neutron has a smaller radius by approximately 15.2% compared to a proton measured in 3-D within the same axial triplet lattice set. The neutron has to reduce in size before a proton can be added to the triplet because the metric diverges outward following the 6/4 axial structure. The proton cannot form until the completion path's distance between all seiches is within a maximal distance between seiches that energy can be transferred across. This phenomenon is evidenced by the neutron lower magnetic moment relative to the proton despite having the same mass and sharing seiches within the same lattice scales. This also explains why some neutrons are often added (costing less energy) before additional protons.

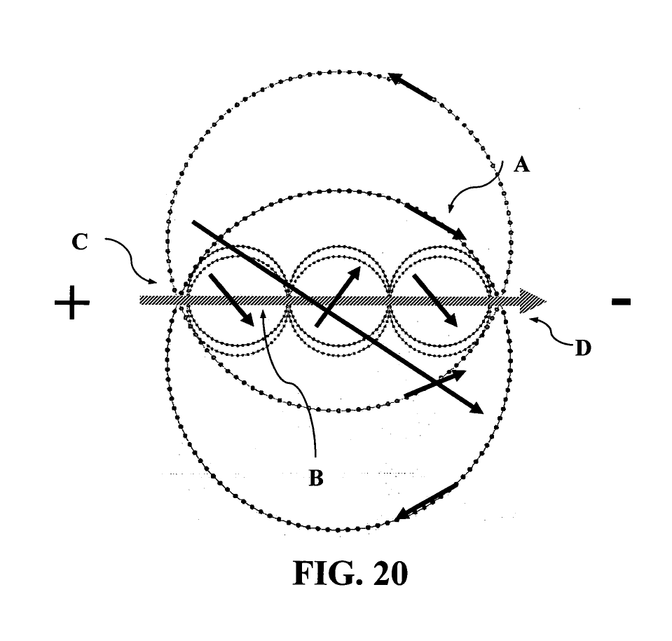

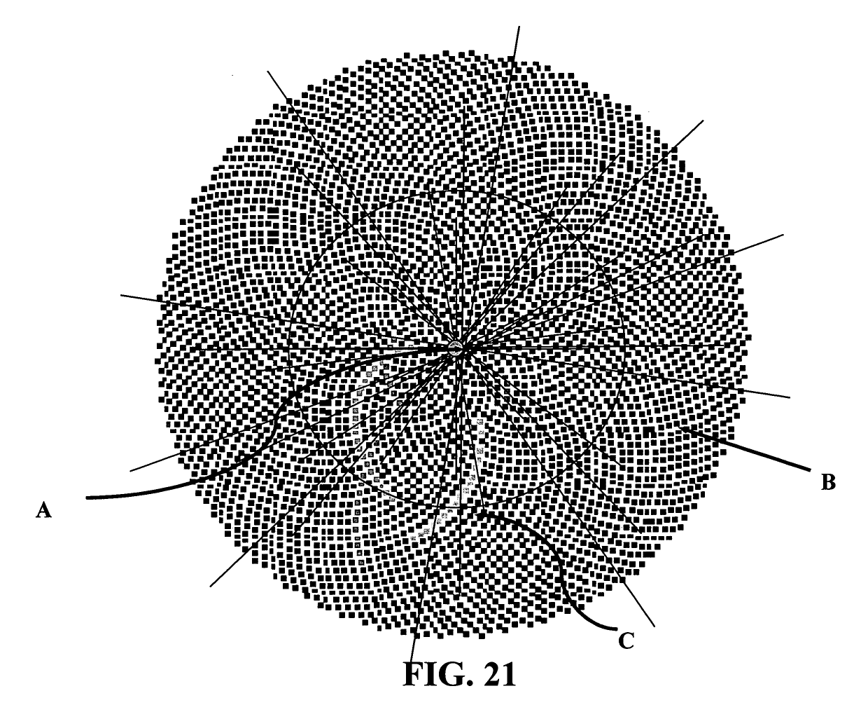

Charge—Charge is the organization of the attractive and repulsive electromagnetic fields associated with the handedness of the rotation combined with the flow through the axial triplets (FIG. 20). Each mass particle has potential for flow and inherent left or right rotation built into it based on the completion sets tilt relative to the radical helicoid.

Electron-proton mass ratio—The Model matches the electron-proton mass ratio to eight orders of magnitude based solely on the Model's torus geometry, high-density lattice sets, field density and intra-mass gap. Mass gap within a particle is based on seiches shared by the 6/4 triplet lattices (Table 10).

TABLE 10
Electron-Proton Mass Ratio
In three Dimensions - Adjusted for
Mass Gap and Seiche Density

| | Electron-Proton | Proton-Electron |
|---|---|---|
| | Mass Ratio | Mass Ratio |
| Known Experimental Measurements | 5.44617E−04 | 1836.1527 |
| Model Predicted | 5.44647E−04 | 1836.0522 |
| Difference: Experiment vs. Model ratios | 2.9786E−08 | |

The calculations for the electron/proton mass ratio confirm that mass is measured in three dimensions, energy transfers in four-dimensions and that the atom is actually a six-dimension structure. The calculations also confirm the structure of mass gap as shared seiches.

Neutron-proton mass ratio—The Model also predicts the neutron-proton mass ratio. The neutron has extra mass because it is tied to the centerpoint neutrino. The intra-mass gap for the proton is 1.6 shared seiches and for the neutron is 1.2 shared seiches (Table 11).

TABLE 11
Neutron-Proton Mass Ratio
In Three Dimensions - Adjusted for
Mass Gap and Seiche Density

| | |
|---|---|
| Experimentally measured neutron-proton mass ratio | 1.001378419 |
| Model predicted neutron-proton mass ratio | 1.001402867 |
| Difference: experiment vs. Model ratios | 2.4414E−5 |

Magnetic moment—The magnetic moment is determinable without perturbation or uncertainty and is calculated to be 1:2197 for the electron-proton ratio based on the raw, unadjusted completion paths and torus geometry in 3-D (Table 12). The energy calculation is then broken down to cover 4-D energy transfer, 5-D particle field generation. The raw geometry of the spindle torus defines the rotation and chirality of the field. Magnetic moment is currently viewed as a rotation based on a 2-D view from outside of the atom. The Axial Model describes the path's full geometry not only representing the completion paths straddling the radical helicoid, but outward and back inward from the centerpoint, with no uncertainty required. The same approach can be taken with any particle.

The actual transfer of energy that generates the moment is based on the completion paths and can be calculated within 6/4 triplets. Further, the density of the shared seiches at either end of the particle lemon is popularly described as the theoretical location of the magnetic monopole. The Model includes that the dipole moment per unit spin angular momentum is twice the unit orbital angular momentum because of the doubled-over completion path in 4-D.

TABLE 12
Magnetic Moment Relative to a Proton

| | Radius* | 3-D | 4-D | 5-D |
|---|---|---|---|---|
| Proton | 1105 | 1.00E+00 | 1.00E+00 | 1.00E+00 |
| Electron | 85 | 4.55E−04 | 3.50E−05 | 2.69E−06 |
| Quark | 325 | 1.59E−02 | 4.43E−03 | 1.25E−03 |
| Pentaquark | 65 | 1.27E−04 | 7.09E−06 | 3.99E−07 |
| Electron Quark | 25 | 7.22E−06 | 1.55E−07 | 3.36E−09 |
| Sub-Pentaquark | 13 | 1.02E−06 | 1.13E−08 | 1.28E−10 |
| Pentaelectron | 5 | 5.77E−08 | 2.48E−10 | 1.08E−12 |
| Sub-pentaelectron | 1 | 4.62E−10 | 3.97E−13 | 3.44E−16 |

*Hydrogen

Electromagnetic attraction—Electromagnetic forces are a result of the six directions interacting- to create the 6/4 field. These directions sweep as they create the 4-D lattice structure. In fact, the sweep of each direction originating from the centerpoint, potentiates the background following the inverse square rules to an infinite distance, thereby creating 6/4 occupiable points at ever-increasing spacing.

The atom provides local disturbance of the background in three ways: (1) the six directions organizing the electromagnetic field, (2) the flow of completion paths and (3) photons. These disturbances to the background are why fields are able to penetrate, potentiate and organize the vacuum of space (vacuum permittivity and permeability).

The electromagnetic field structure is generated locally by the sweeping directions and creates an infinite space of potentiated 4-D points (FIG. 21). The strength and alignment of this field is important for chemical bonding and substantially accounts for the discrete elemental bonding angles. Faster direction rotation creates additional seiche positions further out from the centerpoint, changing potential bonding positions.

The local electromagnetic field facilitates the formation of new centerpoints for new atoms. Because each seiche provides 4 directions/dimensions to a new point, additional local atoms can add additional directions from appropriate angles. The interaction of two or more atoms not only creates a shared potentiated field of 6/4 points, but also provides a catalyst for the formation of new atoms with the addition of two more directions. As with any centerpoint formation, the interaction requires photons of appropriate frequency and amplitude to fill the potential point to become a new centerpoint neutrino and possibly become new matter. This photon energy can be provided from outside sources such as the sun, from local atoms or catalysts.

The electromagnetic field of the atom creates natural angles, distances and positions for atoms to congregate or bond. While many bonds are based on the alignment of electromagnetic fields, some bonds are based on the alignment of six-choose-two hypertube fields. As is demonstrated with the two-slit experiment, the phased interaction of 6/2 hypertubes generated by the photon and particles creates interference patterns.

The strength of the electromagnetic field and resident energy within any given atom naturally organizes the location of neighboring atoms. In DNA replication, local resonance models how DNA repairs simultaneously in sections, rather than sequentially by atom. The resident energy within DNA also provides the underlying energy for replication when introduced to catalysts. When the electromagnetic field is weakened by multiple replications and resident energy is further reduced, replication eventually fails and telemers can shorten. If the field is interrupted, replication is disturbed. The underlying resident energy for each atom has input to the resultant electromagnetic field structure.

Resident energy—The Axial Model introduces the concept of “resident energy.” Resident energy is the level of 4-D+energy within the particle that is not readily visible. It is not matched to mass and is constantly undergoing equilibration within the atom. Resident energy within the atom is measured by the strength of the electromagnetic field.

The disclosed Model allows one to change the resident energy of an atom through the slow, consistent, extended application of narrow-spectrum light to a particle, preferably from a single direction, phase aligned (polarized) and spectral wavelengths associated with secondary intensities of an atom. These photons perform several functions: (1) it fills in for unfilled seiches within the current paths of flow, (2) it adds energy to existing seiches in the completion path and (3) it strengthens fields. Adding energy using one selected frequency ultimately transfers energy throughout the entire atom as equilibration takes place between (a) shared seiches of contiguous particles associated with mass gap, (b) the axial triplets on opposite cones and (c) each axis of the atom through the equilibrating centerpoint. Additional spectral frequencies can also be added sequentially, or concurrently from additional directions as long as the light frequency distributions do not overlap.

Consistent with the Model, increases in resident energy can be persistent. By applying a higher frequency than target spectra, light can be used to enhance resident energy, while applying lower frequency spectra will de-energize the resident energy. Larger seiches associated with lower frequency draw energy from smaller, higher energy seiches.

Secondary-intensity atomic spectra are targeted for changing resident energy because they represent frequencies from particles that have already undergone metric tightening. The highest intensity spectra within the atom correspond to the outermost proton/neutron/electron triplet sets and do not equilibrate energy efficiently throughout the atom. These particles are relatively “soft” in comparison to tighter electron density structures where metric tightening has already occurred. The inner particles have already been tightened and can hold additional energy thereby contributing to the storage of resident energy and metric tightening.

Resident energy at the atomic level is the underlying variable to the coding of DNA at the atom level within the structures of G, C, A and T. There are significant differences in energy levels between any two elemental atoms, and the levels are relatively permanent and therefore generational. At the atomic level, complex field patterns form the successful structure and reproduction of DNA. Resident energy is slowly lost within atoms as cells divide, thereby weakening the fields associated with telemers, where it is thought that this weakening may be associated with the death of a cell.

Atoms within DNA and organic systems can be directly charged or drained of resident energy to enhance characteristics, repair segments, change cellular viability with generational effects on organisms and future offspring. The frequencies that most affect DNA are spectral wavelengths associated with carbon, nitrogen, oxygen, hydrogen and phosphorus. Other tissues and structures incorporate additional elements (e.g., calcium in bone) and will require different wavelengths for different bonds.

Resident energy can be stored inside atoms by manipulation with narrow spectrum light directed at frequencies within the atom's spectral range that are orphaned, that is, separated from the bulk of the spectral wavelengths for that element. The goal is to add non-ionizing, non-heating energy consistently for an extended period of time such as one hour to 30 days or more. Ideally, the element being charged in a small cluster or even a single atom state. Once the energy is added to the atom and the light source is removed, the majority of the energy eventually returns to equilibrium through release of photons from throughout the atom. In general, this process can be accelerated by application of a burst(s) of white light or electromagnetic pulse whereupon the sample will spontaneously release its excess energy or it can release achieving natural equilibrium in food, nutritional supplements and medically therapeutic materials. Some examples of elements and their orphan wavelengths are shown on Table 13.

TABLE 13
Orphan Wavelengths

| Element | Wavelengths (nm) |
|---|---|
| Gold | 662, 736, 768, 827 |
| Hafnium | 460, 762, 724 |
| Potassium | 404, 795, 825, 959, 995 |
| Calcium | 672, 825, 657 |

Using the Model, the method for adding resident energy to an organism for therapeutic purposes is demonstrated. Energy can be applied directly to tissue for therapeutic purposes. Where low doses of light for extended periods of time are not practical resident energy can be added to a surrogate compound and then applied to the patient. Elements or compounds such as gold can be charged. The charged gold then can be ingested, injected, implanted, or applied topically to add resident energy to local tissue.

Heat—Heat disrupts the transfer of energy within the completion path. The physics of phase transitions includes that heat is disruptive to flow as demonstrated by the tendency of a magnet to lose strength as it is heated, with total loss occurring above a certain finite critical temperature. The conclusions also match observations associated with “frozen light” experiments where light was selectively used to keep the atom between energy states to inhibit flow and photon release with a specific spectral frequency, thereby obstructing another frequency.

Maintaining sequential timing on completion paths maintains energy flow. As with heat and radioactivity, decay is a means to reach equilibrium. The decay components are based on the particular particle and flow paths in the atom. In the case of heat, photons are expelled, because energy is lost when the seiches are not able to accommodate the additional energy. In the case of radioactive particles, an entire triplet or cone set can be lost (alpha particle), particularly those occupying outside (higher level) positions.

The Photon

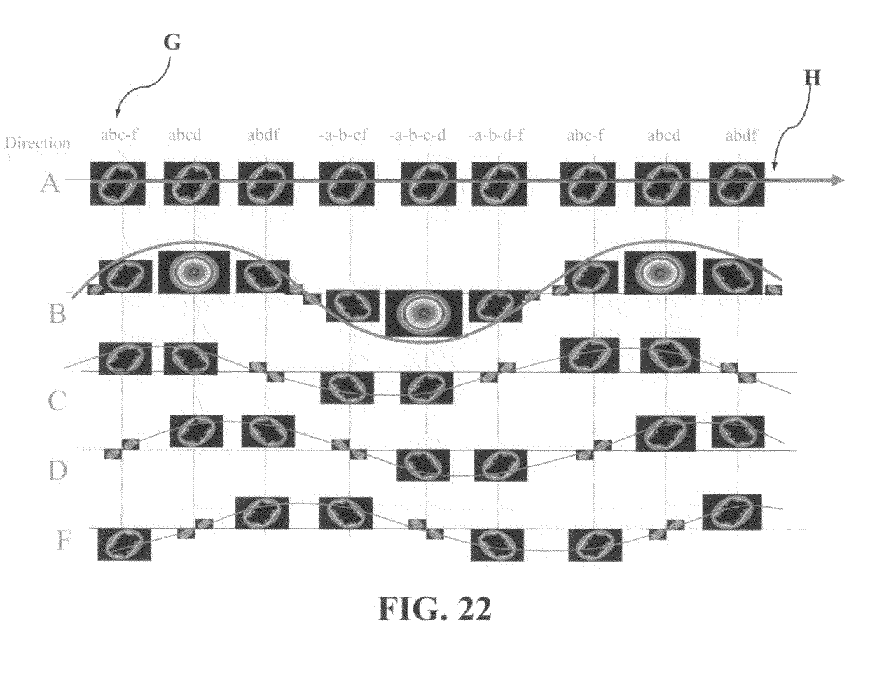

The photon is a 5-D energy packet whose frequency, amplitude, and helicity are determined directly from the geometry of the completion path from which the photon emanated and the excitation of rotation of each of the five directions. Changes in direction rotation cause photons to be released. If one of the directions does not match the completion path, a photon cannot be emitted. The photon has a dynamic structure based on five wave variables, one of which has lost its particle-based periodicity and thus travels at the speed of light (FIG. 22). Each seiche is shown to have one direction with no periodicity and three waves with the periodicity consistent with the particle from which the photon was generated. The fourth dimension with no periodicity is represented as the time variable in Special Relativity where the forward motion is represented as “c,” or the speed of light.

There is a ground state frequency associated with each particle and related triplet within the atom. The relative radius of the particle generates the periodic character and frequency of the photon. As previously discussed, protons and neutrons have different relative geometries even though they share the same lattice triplet scale. This is also true for particles across the centerpoint, where extra tightening is required to continue to tighten the lattice to add additional proton/neutron sets. The Model's geometric character provides utility in that the position of each particle on each axis can be determined along with its light signature.

The photon is a 5-D energy packet that is released from the completion paths that has had one of its five dimensions changed sufficiently such that the periodicity is lost for that dimension and the particle then travels at the speed of light. The remaining dimensions do not change their periodicity and travel with the same periodicity as the originating completion path. A photon emanates directly from a seiche when one directional variable changes for the individual seiche or for the atom. The photon follows the same sequential path of the three lattice sets of the completion path from which it is released. This reveals that the structure of a photon has four directions of wave periodic influence on its trapped energy level. The 5^{th} direction has lost its period and results in the free photon traveling at the speed of light, following a straight path. The photon is only emitted when the atom reaches the next level of alignment between directions to create a 6/4 position (accounting for the discrete wavelengths emitted by particles and elements).

Consistent with the torus and completion path from which the photon escapes, the photon will either be auguring (sequencing) in a left or right rotation, consistent with the periodicities of the originating completion path. Completion paths and photons always share three lattice sets. In some cases, the triplet exhibits using directions in two or three of the lattice sets. (e.g., the triplet set ABCD, ABCF and ABDF uses direction A in all three paths and D in only two paths). Directions involved in all three-lattice sets have larger amplitude in one direction than where the direction is involved in only two lattice sets.

Rotating plates—A visual analogy to the interaction of rotating directions/dimensions can be constructed using two spinning pie plates, each with a single hole in the same part of the plates, near the edge. Rotating the plates in opposite directions only allows light to pass through the hole (similar to creating a 2-D seiche) where and when the holes overlap; one plate can spin at exactly twice or three times the speed and the same position and open space appears. While each increase in plate rotation speed adds energy to the system, the alignment of the 4-D spaces occur only in whole number of spins. Using four plates spinning in opposite pairs on the X and Y-axes resembles the formation of a 4-D seiche.

Quantization of light—Light is emitted in quanta because the rotating directions have to achieve alignment and reestablish the completion path for a photon to be released. If completion path flow is interrupted, disrupted or the formation of 4-D seiches does not occur, a photon cannot be released. In the important case of excitation states, where individual directions have faster rotation (in whole steps), emitted photon will have the energy difference between the excited state and the rest state when the flow renormalizes.

Seiche sequence and helicity is the same for all completion paths within a particle. The tightness of the radius of periodicity also matches for all three lattices paths. Photon energy is absorbed and re-emitted at discrete frequencies matching the radius of the particle. Additional matching photons fill in empty seiche positions and tighten the metric. Each additional absorbed spectral photon strengthens the structure and puts more energy into the system by (1) strengthening weak 4-D seiches, (2) filling more completion path seiches and (3) tightening the metric. This additional energy, over time, adds energy to all particles throughout the atom using equilibration through the centerpoint.

Fine and hyperfine structures—The quantization of the photon energy is the result of the specific geometries associated with completion path sets. Each direction sweeps independently. The Model includes that a single direction is part of two or three seiche positions and this results in there being more than one possible rest value within a single completion path or completion set to realign 6/4 seiches. As shown in FIG. 14, where the three completion paths cross there are two intersections for each path providing two points where the rotations within a single 6/4 lattice set can rejoin a seiche theoretically providing the fine structure wavelengths (between points on 6/4 lattice set ABCD). Based on the model and projecting forward, the hyperfine structure is revealed when the three sequential positions within a completion path (using three 6/4 lattice sets) provide unique resting points for the rotation of the direction to come to rest (between ABCD, ABCF or ABDF within the triplet).

Using the plate analogy, the position of the photon and alignment of the directions is slightly more complex in that on any given plate, there are actually two or three “holes” that can be used for alignment of a discrete 4-D space as described by the wave Model. As a single direction speeds up or slows, the energy level absorbed or emitted from the completion path is dictated by the new plate alignment. Because there are several options (multiple holes per plate) for that alignment, frequencies that are emitted have small differences in spectral energy. These differences can account for the fine structure and Lamb shifts from the basic frequencies associated with excitation states. The Lamb shift is likely associated with the difference between two seiches within the same lattice and fine structure is likely represented by the energy difference between two seiches in different lattices (1/(n⁴)² scale).

A photon is seen as both a particle and a wave because different measurement techniques yield different observations about the same structure. A photon's energy is transferring from seich-to-seiche as modeled by the Axial Model, acting as an energy packet. However, when the waves of a photon are phase aligned, the interference of the directions between the point sources is the dominant visual signature. The photon energy is still transferring from point to point but is not visible as it is made up of a collection of 4-D waves. The same is true for similar particle experiments.

Slit experiments—What is important about slit and a delayed choice experiment is the concept of phase timing. If the constituent waves within a photon or particle are phase matched (6/2 or 6/4) upon passing through two parallel slits, the waves will interfere creating the well-known interference pattern. If waves are not in phase, they will not create an interference pattern. When light is polarized, it is sorted for phase. If the slits are orthogonal, there is no visible interference. If the light is filtered through orthogonal slits again, phase alignment is returned and the interference returns. This experiment demonstrates the intensity of interaction between phase-aligned 6/2 structures. The propensity for directions to mutually interfere, particularly when phase aligned is very important to understanding the interaction of particle waves.

When a particle passes through a slit it only acts as multiple photons because the 4-D seiches are in phase with each other and thereby create interference patterns. In actuality, the photon or particle only passes through one slit. The interference to the background passes through two slits. Further, since the 6/4 axis is self-referencing, any portion of the particle or flow path that is removed while going through the slit, will be restored. The interference patterns are generated by 6/2 hypertube alignment and 6/4 alignment; the 5-D photon remains intact.

A revised equation for the excitation of atoms and the resultant photons is based on the following parameters: (1) the excitation of specific directions included within the triplet (within a completion path, five variables for speed of rotation of the independent directions are considered, A², B², C², D² and F² where one direction has lost its periodicity) and (2) the relative radius of each particle within each triplet. The Model shows that the diameter of the atom does not need to change as the frequency of one direction is changed. This allows the excitation rules to apply to many-electron atoms and not just single-electron atoms like hydrogen.

When a photon is released, the completion path energy transfers from seiche-to-seiche indefinitely as the particle travels through materially “empty” space. The availability of seiches may change, but the speed of transfer between any two points stays at the speed of light. This models the causal structure for the observer always measuring light at a fixed speed regardless of the speed of the source. If the local electromagnetic or gravitational field changes the position of the next available point, light bends in a similar fashion to the closed loop in a particle.

Since the completion paths are the surface of the torus, photons are released from the surface. For example, when a change in direction occurs photons are lost predominantly from the end of the axis as these particles have the largest distance to cross between seiches. In more dramatic cases, the release of multiple-seiche energy is rapid and appears conventionally as fire—a photon release of energy on a large scale with two or more direction parameters changing at the same time. Paramagnetic structures such as oxygen can facilitate such changes.

Spin-spin—The inherent periodicity of the six directions and the 15 6/4 axes reveals that a 6/4 axis set on one side of the centerpoint is matched by the set on the other side of the centerpoint. As a result, the actual lattice set for the neutron starts at the centerpoint and appears to return through the centerpoint twice for every single sweep of a dimension/direction. This double motion is why the spin-spin and the spin-orbit ratios are close to 2:1.

This doubled geometry is required in 4-D as each direction is independent and as each wave crosses the axis (e.g., sine wave at {0, 0} and {0, X}) one point goes through the centerpoint and the other position is really passing near the centerpoint. A single particle completion path of 108 is actually two loops of 54 when plotted in the context of all four dimensions as a result of the 4 periodicities interacting. The torus is tied to the centerpoint at one end of the lemon and one intersection at the top of the lemon. This path cannot be accurately rendered in 3-D, and for simplicity and accuracy is represented as single 2-D circular loops of 108 seiche points which technically more accurate. This simplification does not change the number of seiches in the circle lattice, angles of lemon intersection, torus solutions or relative scales of particles. However, the geometry does naturally provide the 5-D density that is commonly described as the location of the density associated with the hydrogen atom, opposite of the electron's position. In larger particles, the centerpoint is a neutrino.

The ratio measured experimentally is slightly above two because the intersection of the three completion paths straddles the radical axis. Therefore, to complete a full path, returning to a measurable single starting point, the calculation must go to the next lattice point beyond the center axis (approximately 1.666° for a neutron or proton and 5° for the electron, plus or minus one triplet seiche).

Einstein-Podolsky-Rosen—In a normal collection of atoms, the handedness of light appears random. Within a specific atom, however, the handedness of the particle and the light it emits is determinable and is solely based on the triplet and particle from which the photon was released. It is always determined at the source.

Particle Influences

There are a select group of influences that electromagnetic radiation has on an atom. These include, excitation, stimulation, metric tightening, chaos, cooling and their respective opposites. Each influence is achieved through different techniques, and they are broadly defined below:

Excitation—The process of adding energy to an atom such that the complex interaction of at least one of the six independent directions is changed and the seiche paths are altered. This is a short-term effect as the atom seeks the lowest energy state unless acted upon by an outside force. It does not contribute to resident energy significantly.

Heat and chaos—The process of adding broad-spectrum radiation or excessive amounts of narrow wavelengths to an atom that disrupts flow and causes a cascade of photons to be absorbed and reemitted with no residual increase to the completion path energy of the atom. Heat actually causes the reduction of completion path flow and charge for the particle. Plasma takes this to the extreme where without flow and shared seiches, electrons are released.

Stimulation—The process of adding single wavelength energy at high intensity to an atom usually matching its most intense spectral line(s) to add and release photons usually of very short duration measured in seconds or parts of seconds. These involve rapid changes in energy but have little effect on resident energy levels as excited atoms seek equilibrium rapidly.

Laser “cooling”—Adding single wavelength light to an atom at intensity sufficient to prevent the atom from reaching a stable excited state prevents photons from being emitted and halts completion path energy flow. This technique has the effect of keeping five of the atom's six directions (or four of five triplet sets) from flowing. This is not truly cooling, rather, total disruption of completion path flow such that the atom exhibits no spectral emission, appearing consistent with being very cold. In fact, the atoms are not truly “cold.” This technique does not allow for equilibration of the atom and therefore does not significantly add to resident energy.

Newly Discovered Influences on Particles

The Axial Model reveals additional influences on particles that provide useful and novel applications for photons and particles.

Resident energy—A feature introduced by the Axial Model is that the resident energy can be increased by applying single wavelengths light of low intensity matching secondary-intensity wavelengths within the atom over extended periods of time (measured in hours, days, weeks and months) at energy levels that do not add heat. This causes the atom to collect energy and which equilibrates throughout the atom over time. Changes to resident energy are measure through changes in field strength of the target atoms. Low-energy applications of light changes resident energy significantly. Resident energy can unstable for a period and can spontaneously be released in a burst upon application of appropriate electromagnetic stimuli (e.g., carbon nanotubes exposed to camera flash).

Broad frequencies of energy, such as white light, add heat/chaos and consequently do not contribute to resident energy, but can serve to add gentle disruption to directions required to create/steer new mirror path formation once the foundation particles have had resident energy added and metrics tightened. Local seiche disruption is important to changing the paths of trapped energy. This reaction does not require a lot of power, but does involve perfect timing. One photon delivered to the shared seiche at the right time may supply sufficient energy to change the path of flow to create a mirror particle, e.g., to add proton. Too much energy changes more than one Julia variable, which is likely not to result in a new completion set. If electromagnetic stimulation is added too swiftly or through use of an appropriate electromagnetic wave, the built-up resident energy can be spontaneously released.

Elemental simulation—Another feature revealed by the Model is that the spectral energy of an individual element (e.g., oxygen) can be simulated within cells or an organism by adding multiple narrow-band frequencies of light that match the element's spectra, preferably individually and sequentially. These select frequencies can be used to add “energy of specific elemental wavelengths”, manipulating DNA replication and protein expression, DNA repair and replication at the atomic level. They can also contribute excited state energy to redox reactions to manipulate reaction characteristics and character of product.

Metric matching—Another feature of the Axial Model is that the axial triplets and resulting chirality can be understood and redeployed as a tool for deterministically changing bond potentials between elements and compounds. Matching the lattice tightness, helicoid character and directional energy will allow direct manipulation of bond potentials for chemical manufacturing and drug discovery.

Metric tightening—The Axial Model reveals that sufficient energy applied from a narrow source light similar to resident energy, over time, tightens the metric. Metric tightening is an extension of resident energy where additional particles are added or taken away from a target atom.

Single-handed photons—The Axial Model provides a method for directing the emission of a single photon of known chirality from an atom for applications in computers, telecommunications and encryption. Different frequencies can change the strength of bonding on select triplet axes. Specific chemical and biological reactions can be controlled knowing the location of each particle on its specific axis, its chirality and wavelength. Adding single-handed photons to a chemical reaction serves to align the radical axes, enhancing bonding.

The Axial Model includes that there are two primary contributions that an atom can offer a redox reaction: field organization and energy transfer. First, an atom can contribute its organizational structure, including its axial field structure, strength, and chiral organization. This organization is the foundation for elemental bonding.

Second, atoms can exchange energy. Energy exchange can be accomplished through the direct transfer of photons or equilibration of energy within the 6/n field structure. For example, alignment of 6/2 structures between atoms holds two of the six directions in synchronous alignment while still allowing complex rotation of the remaining four axes.

Energy exchange is facilitated when the field structures are matched for chirality and frequency, an indication of metric matching often requiring one atom to tighten and the other atom to relax. Higher energy systems are tighter and lower energy systems are looser. The potential energy of the bond is stored in the atom.

The Model is useful to determine the specific axial bonding sites between elements and the frequency and the metric matching required to complete a redox reaction.

Expansion of Ten Primary Cones—The Hierarchial Structure Formation of Atoms with Increasing Mass

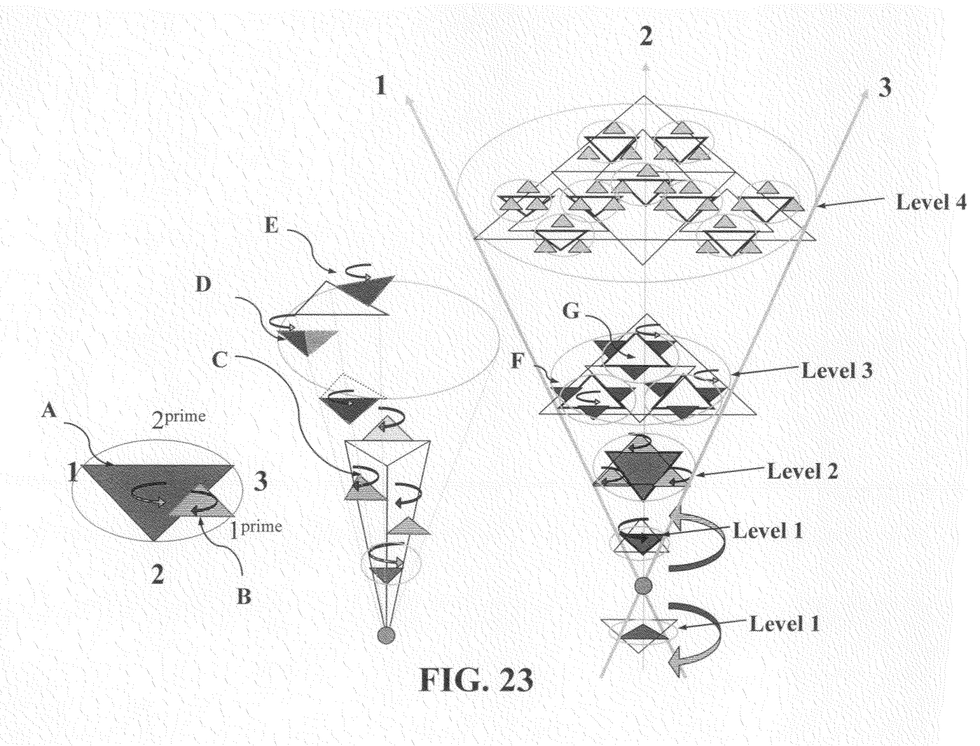

The base structure of the atom is ten 6/4 cones on the five radical axes and is defined within the 6/4 lattice (FIG. 23). Each cone can contain a neutron, proton and electron aligned on the radical axis, filling the four-dimension space between the electron and the centerpoint.

As the metric paths fill and tighten, the next sub-cone or particle position can form farther out from the centerpoint, in effect branching within the context of the base cone. Two of the three original triplet axes (2, 3) and a new, resonant third axis (1^{prime}), across from the original third of the triplet axis form the new triplet.

Using the Model, the cone/sub-cone formation is regularized and the causal structure for particle growth is determined. Sub-cones have an opposite rotation sequences and flow paths versus the cone level directly preceding them. The entire cone and sub-cone set stays within the triplet cone area, forming a single large cone from each of the ten primary cones.

The cone is more than a visual metaphor; it provides the organizational limits to the position of the radical axes in larger atoms. As shown, the cone is formed by the triplet axis. As the cone gets larger, the sequence of the axes changes as the next subcone uses two of the axes and the prime (negative) of the opposite 6/4 axis as part of the completion set. Between sets of triplets, contiguous cones actually overlap, however, because 6/4 completion path is based on independent sets of directions, they never intersect.

Within each of the ten primary cones out from the centerpoint there is an additional level of three subcones (level 2) and out again from the three sub-cones is another level of sub-cone positions forming nine new sub-cones (level 3). As the cone axis rotates, the next level sub-cone “gears” in an opposite direction from the level below it. As the 6/4 base cone axes (e.g., 1, 2, 3) rotate in sequence, the sub-cones form using the related axes set (2, 3, 1^{prime}) and, therefore, have an opposite sequence. Each base cone sub-divides to three sub-cones. Each of the three sub-cones can further divide into three subsequent sub-cone sets (a total of 13 stable particles per cone).

There is a total of 13 stable cone and sub-cone positions for each of the ten base cones, yielding a total of 130 potential positions for protons and 130 potential positions for neutrons; a total of 260 potential stable particle positions. On an even larger scale, there are an additional 27 sub-cones per base cone on level four (270 total additional cones or 540 potential particles for level 4); these are not stable structures as they describe extended axial structures of radioactive elements larger than uranium.

Radioactive decay is the result of the separation of a particle completion path from synchronization (timing) with the rest of the atom. This results in particle emission, axial triplet emission (alpha particles) and radioactive decay. Unstable extended triplets (levels 3 and 4) of neutrons and protons can “lose timing” with the centerpoint. Timing loss is when energy transferring through the completion path returns to the original centerpoint or shared seiche position and the centerpoint/shared seiche is no longer there. The particle is summarily released from the atom on a vector. This loss of timing can be from disruption of the flow path, hitting and moving the centerpoint, or from ended axial triplet particles not being able to complete the path of flow in synchronization with the rest of the atom.

Full triplet and cone emission is exemplified by uranium fission. Laser-induced fission was observed at the VULCAN laser facility in 1999. It has been demonstrated experimentally, that fission of uranium produces a double-headed asymmetric yield distribution of fragments, with maximum fragment yields averaging mass of 95 and 140. These measured values are predicted by the Axial Model and cone pair cleaving. Cone pairs are bundles of triplets. Cleaving occurs in uranium when three cone sets (six of the ten total cones) break from the remaining two cone pairs during uranium fission. For uranium, the Model shows that each of the ten primary cones has an average mass of 23 to 24 neutrons/protons per cone so that cone pairs have a mass in the ranges of 95 and 140 when broken into cone sets of four and six cones.

Cones cleave in triplets, similar to alpha emissions that comprise the ejection of a simple axial triplet, most frequently from levels three or four, which is where the Model includes that extended axial triplets are most easily lost. This is consistent with the concept of seeking the lowest possible energy state when split.

The conceptual equator and axial triplets provide the conceptual framework that a heavy nucleus deforms and spontaneously splits apart in higher mass atoms in a high spin state (e.g., heavy actinides as well as some rare earth elements).

More Magic Numbers for Protons and Neutrons

The Axial Model described herein offers an explanation for the build-up of the atom in layers of ten cones and why atoms appears to be stable even when some of the axial proton/neutron pairs are not completed. The major reason for this is that when a single hyperplane wave direction is “increased in energy”, it only affects eight of the ten cone sets providing important tightening asymmetrically to the atom. The atom may have reached its energy balance at level 1 with neon, yet the atom has insufficient directional energy and lattice tightening to completely fill level 2 sub-cones (e.g. Argon 18 and Iron 26), often the case above the mass of neon. Numerous larger relative radius lattice sets with 108 seiches provide structure for neutrons and protons. The model describes two levels of cone completion based on the scale and position of the next particle to be added to the cone. Major cone levels are full at the 10, 40, 70, 100 and 130 levels. Minor cone levels are in sets of ten corresponding to the number of base triplet cones in the atom.

Electron Orbits

The Model defines the position of the electrons using five dimensions within the triplet cone. Electron orbits do not intersect because of the 6/4 lattice configuration and because each triplet naturally has a different path through the atom. Further, each successive particle within the cone has a different scale for its completion set assuring particle paths will not intersect short of catastrophe. Finally, the neutron, proton and electron share seiches, tied together, ensuring cooperation.

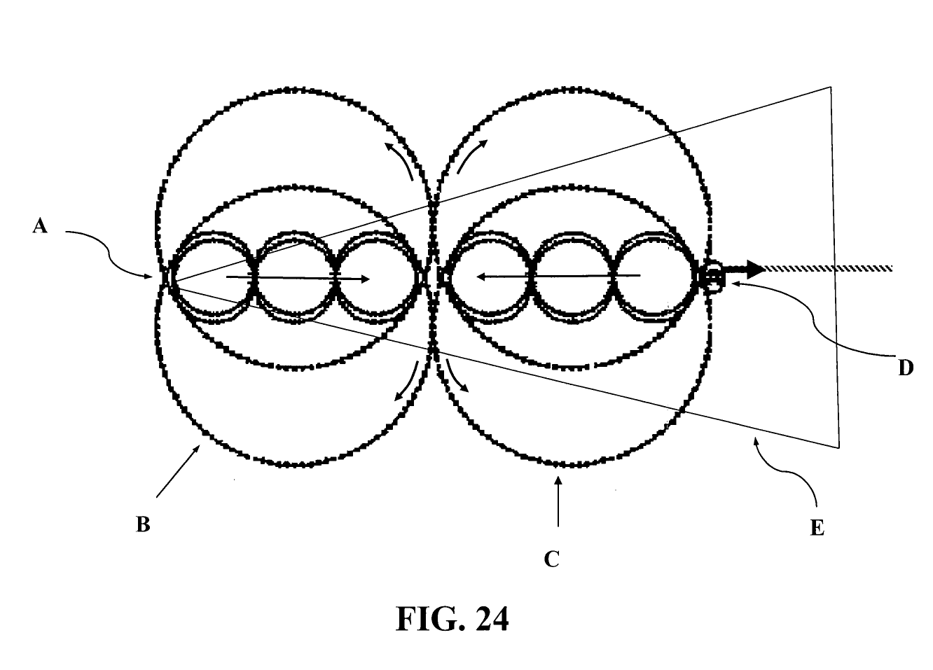

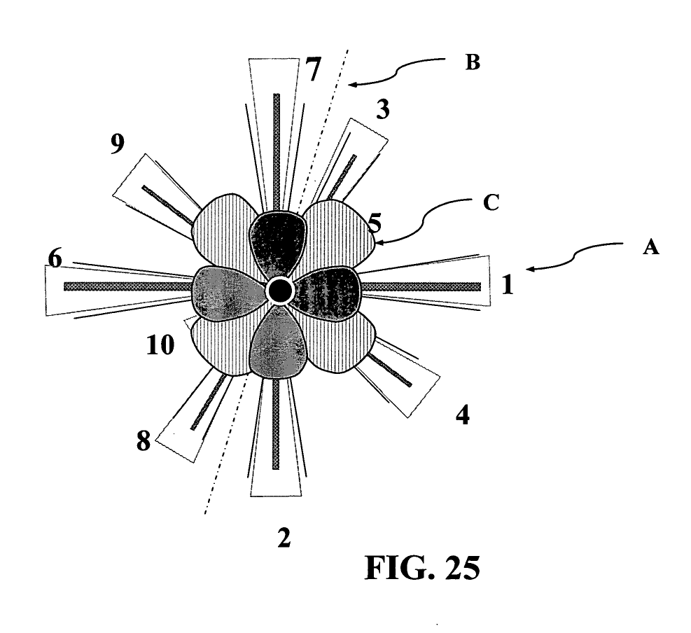

The complex 6/4-D structure defines the “cloud” movement of the electron at the end of the proton/neutron axis (FIG. 24). The electron is a major confinement, 5-D particle. The flow of the electron is outward and mirror opposite that of the proton, just like the neutron is mirror opposite the proton. The distance and position from the centerpoint for the electron depend on the five directional variables of the neutron and proton. The Model takes into account the x, y and z components of orbits (FIG. 25). The electron is released when the three shared seiches with the proton (one for each completion path) is no longer occupiable.

Singlet atoms reflect changes to the orbits of the electrons. In the case of singlet oxygen, the eighth neutron-proton pair has switched sides of the atom and the atom is now oriented on only four radical axes instead of five. This causes the atom to become magnetic with destructive single bonding instead of paramagnetic requiring double bonds.

Pauli exclusion principal—The model provides the natural underlying structure of the Pauli exclusion principle. The six-choose-four axes are self-referencing and do not cross each other naturally Crests and troughs are involved in the formation of 6/4 axes and provide discrete lattice spacing associated with separations of individual 4-D lattice crests and troughs. Further, the expansion of the cone usinig discrete particle scales also ensures the particles do not collide with other particles from “above” or “below”. Each neutron completion path has its own seiche position to enter and exit the 6-D centerpoint.

Tightening Metric

As energy is added to seiches and completion paths, they become smaller and tighter. The Julia fractal is an iterative (similar to “curled-up” language) complex system that has tremendous energy-holding power in four dimensions. Adding energy to an individual seiche, completion path or changing excitation levels does not affect the 3-D measurement of mass, however, as the seiches network adds more energy, the local seiches get smaller and tighter. This explains the recent observation that adding a lambda 7 particle to a lithium nucleus tightened the radius of the atom by 19% (Tanida, K., et. al., “Measurement of the B (E2) of Lambda 7 Li and Shrinkage of the Hypernuclear Size,” Physical Review Letters, 86, 1982 (print issue of Mar. 5, 2001).

Protons are not all the same size or energy level. All particles of major confinement with 108 seiches are described as a proton, regardless of the radius of the lattice scale from which they were formed. The only limiting factor to successful determination of a completion path is a maximal distance between seiches that energy can be transferred across. Within a tightened metric, a new particle can form when the scale of the most recently added particle has reduced to the point where the next largest radius of given lattice points can form, not exceeding the maximal distance rule. The new 108-point particle will have the same three-dimensional mass value despite having a larger relative radius. This provides for nesting of smaller and larger protons within an atom, while always measuring mass of each proton as one.

The available sets for this lattice structure provide a quantization of the relative scales of all protons within the atom. The relative radius scale of the lattice varies with each triplet set. A neutron/proton particle set must reduce in size to add additional sets on a cone. In sequence, to add another particle set, the metric has to tighten further. The tightening structure does not collapse to zero since there is natural lattice spacing within 6/4 triplets. While massive amounts of energy can be stored as structure tightening is close to infinite, lattice spacing within the particle is maintained.

New completion sets matching the lattice count and relative radius of the proton occur at discrete scales. Table 14 shows the relative radius of each completion path that makes up complex spindle torus protons of varying relative radii. Each newly added proton or neutron has its own set of sub-particles, including quarks and pentaquarks. Not all paths with 108 lattice points can be protons because some do not have the required substructure set confinement parameters (e.g., radius 2210). Once again, the relative radius of a proton does not change its 3-D mass measurement only the 4-D energy level as the radius tightens.

The Model shows why simple quarks can be shown to have multiple levels of energy yet geometrically; they can be substituted within protons because the distance between seiches fits within the proton. Further, the Model demonstrates why the atomic table shows atoms generally shrinking in radius as one moves to the right on each period, increasing significantly at major cone levels and slightly at minor cone levels.

TABLE 14
Proton Scale Formation Sets - Torus Radii “r”

| Triplet | Pentaquark | Quark | Proton | Proj. Elemental |
|---|---|---|---|---|
| Number | 36 seiches | 60 seiches | 108 seiches | Scale |
| 1 | 65 | 325 | 1105 | H |
| 1 | 145 | 725 | 2465 | He |
| 2 | 185 | 925 | 3145 | Be |
| 3 | 195 | 975 | 3315 | Li |
| 4 | 205 | 1025 | 3485 | c-12, Singlet O |
| 5 | 265 | 1325 | 4505 | 0, Ne |
| 6 | 305 | 1525 | 5185 | K |
| 7 | 365 | 1825 | 6205 | Ca |
| 8 | 435 | 2175 | 7395 | |
| 9 | 445 | 2225 | 7565 | |
| 10 | 455 | 2275 | 7735 | Ar |
| 11 | 485 | 2425 | 8245 | |
| 12 | 505 | 2525 | 8580 | |
| 13 | 545 | 2725 | 9265 | |
| 14 | 555 | 2775 | 9435 | |
| 15 | 565 | 2825 | 9605 | Fe |

Chemical bonding—The model projects that there are two primary bond structures between two atoms, (1) bonding associated with hypertube 6/2, 6/4 or 6/6 structures, and (2) axial bonding associated with aligning triplet axes. Bonding associated with center-faced cube structures can be projected by the model as based on sharing six choose two hypertube organization. Axial bonds, where the metrics of two triplets are aligned to match helicity and tightness (or multiples thereof) are seen in molecules associated with carbon or oxygen. Changes in resident energy and metric tightening facilitate the alignment and bonding of elements. The energy that is associated with a chemical bond is derived from the extra energy that is added to the atom to complete the bond.

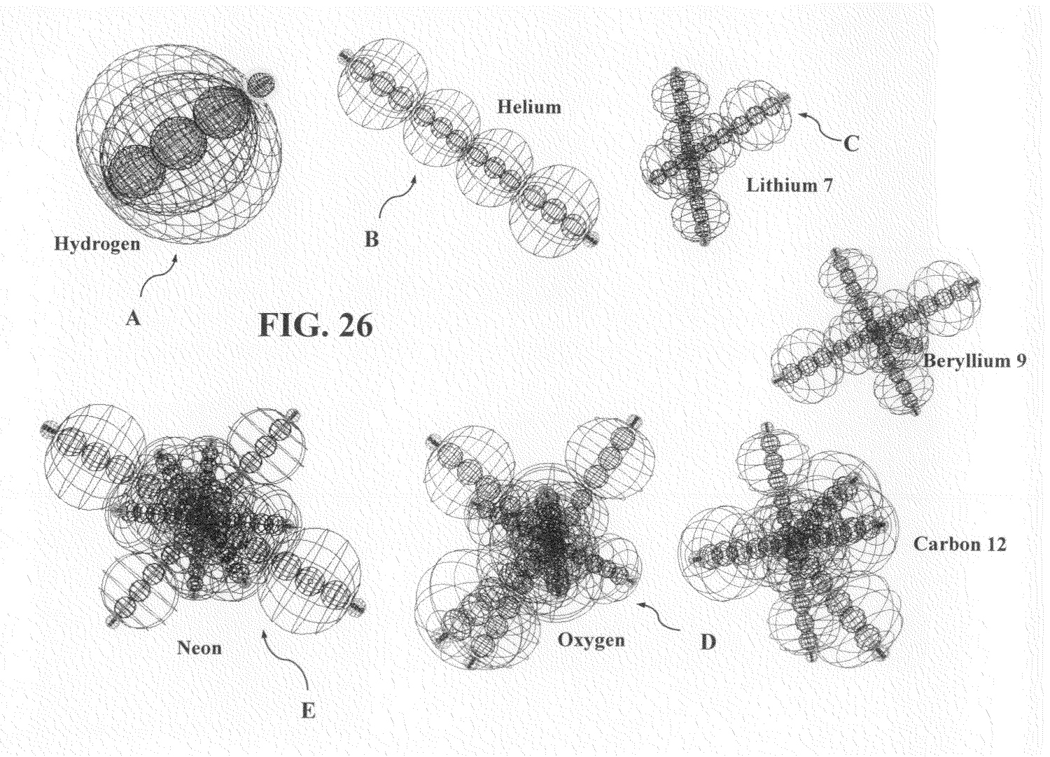

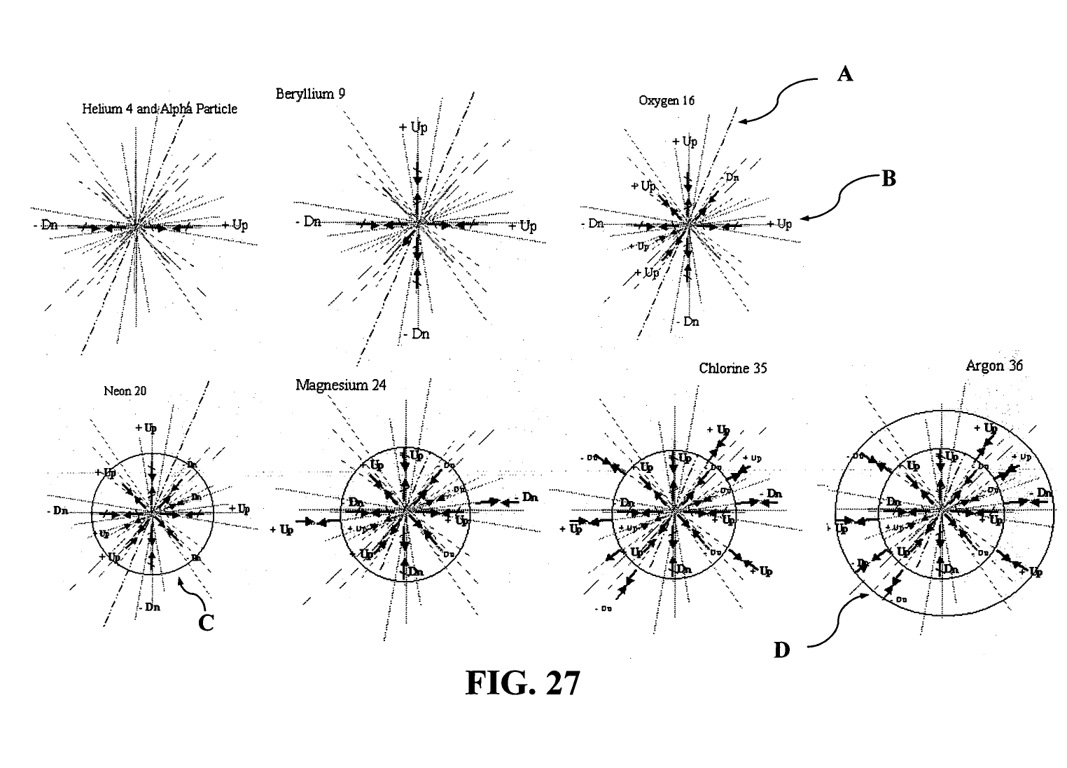

Atomic Models—Selected atomic models are prepared and drawn using the methods and concepts disclosed herein highlighting the relative positions of the protons, neutrons and electrons. Several atomic models using representations of spindle tori are shown in FIG. 26. In FIG. 27 additional atomic models represent the atom in two dimensions, highlighting the relative positions of particles within the atom's structural levels, a descriptive and useful map for representing particle position.

Gravity

Gravity waves are hyperplane waves generated by the completion paths as the cyclic flow disturbs the background. Gravity waves from particle mass are the result of trapped energy pulsing in sync through the atom, out from the centerpoint and then back inward, resulting in a cyclic disturbance to the hyperplane background. Gravity waves associated with mass travel at the speed of light because of the synchronous flow that creates mass, light and fields. The trapped energy path pulse has the effect of stimulating the hyperplane grid in waves.

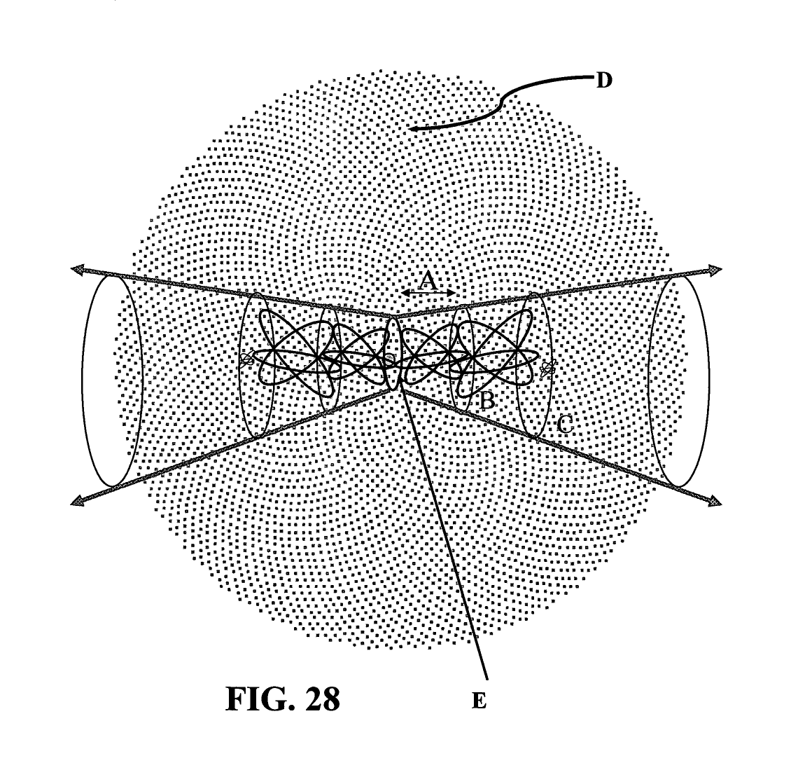

Gravity waves are generated by each particle within 5-D cone and by the flow of trapped energy within the completion paths of each particle. In the case of 1 protons and neutrons, completion path energy travels traveling synchronously through 108 seiches, regardless of the particle's relative radius (FIG. 28).

Since the three completion paths within a single particle use the same three 6/4 lattice sets to transfer energy within its trapped path gravity acts with a unified motion within each 5-D cone. Since gravity waves are generated by the transfer of energy from seiche to seiche, gravity waves associated with mass travel at the speed of light. Logically, it would be reasonable to assume that the gravity generation at the atom level can be disrupted by the disturbance of the individual directions such that the completion paths were unable to complete, effectively halting flow through the completion paths, in a manner similar to “laser cooling” used in the frozen light experiment, described earlier. Each completion path and each cone generate its own gravity pulse, which explains why gravity has been described as a 5-D (or 10-D) phenomenon.

This same field generation is associated with bonding and energy transfer between atoms and molecules. Local gravity field disturbance also helps to create the localized “resonance” within the atom facilitating new particle formation on the same side of the atom before completing respective axial triplets across the centerpoint (Hund's rule).

Gravity scale—The scale of gravity is miniscule compared to the scale of the electromagnetic field, with gravity measured at an incredible 10E-40 in scale relative to the electromagnetic field. There are three sources of field disturbance by the atom: first, the pulse of gravity as described above, second, the atom's six directions potentiate the electromagnetic force which emanates from the neutrino centerpoint outward at infinite distances and third, the photon. To compare the scales of the electromagnetic field to the gravity field, the scales have to be matched, that is, the torus can be inscribed within a cylinder. The complex cylinder math is shown in Table 15.

TABLE 15
Volumes of Complex Cylinders (8)

| Dimension n | Cylinder Volume |
|---|---|
| 3 | 2πr(r + R)² |
| 4 | (8/3)πr(r + R)³ |
| 5 | π²r(r + R)⁴ |
| 6 | (16/15) π²r(r + R)⁵ |

Where r is the radius of the torus tube and where R is measured from the radical center of the torus to the outer rim.

Gravity waves are generated by a finite number of seiche positions within the particle confined by a cylinder (e.g., the proton has three completion paths of 108 seiches, each path using three 6/4 lattice sets for a total of 972 seiches per proton). The electromagnetic field is generated from the centerpoint. In the case of the confining 5-D cylinder for a hydrogen proton, the height is r=1105 and the cylinder radius is R=1492 (65% overlap torus). As a 5-D cylinder (to match the torus) the electromagnetic field seiches for hydrogen within just the cylinder are 2.20404E+19. The total seiche count for the proton particle is 972 ((3*108)*3); the resulting ratio of the seiche counts of gravity to electromagnetic field measures exactly 4.4100E-17 using 5 dimensions.

However, the electromagnetic field is generated from the centerpoint and the gravity wave is generated by the completion path seiches at some distance from the centerpoint. This distance can be generalized as “x” or a multiple of “x” from the centerpoint. The field strength of the any seiche position on the completion path relative to the centerpoint is weaker than the centerpoint by a ratio of 1/x², no matter from what position or distance it is measured. The strength of the electromagnetic force to any gravity seiche then is x². Logically then, the ratio of the hydrogen proton gravity wave to its electromagnetic field is (4.41E-17)²=1.95E-33 in 5-D. The measurement for hydrogen is much higher than theorized today, This is explained using an analysis based on the concepts promulgated in the Axial Model.

Further exploration surprisingly revealed that the scale of gravity to the electromagnetic force is not the same for identical particles in different elements. The gravity to electromagnetic field scale for outer protons in heavier elements such as carbon is actually lower than helium (the first atomic triplet) because the relative radius of the carbon atom, 3,485, creates a cylinder volume of 2.17E+22, and an adjusted gravity to electromagnetic ratio of 2.01E-39 for the outermost carbon proton. For Iron, the relative radius for the outermost proton is 9,605, creating an adjusted ratio of 1.05E-44. The true ratio for the iron atom between the innermost triplet (He), 1.28E-37, and the outermost and largest proton 1.05E-44, creating a calculable value for each of the triplets as shown in Table 16, with an average value for all iron triplets of 9.39E-39. Hydrogen is excluded from the average, as it would represent double counting of the first triplet and is not representative of a 6/4 structure.

The scale of gravity force to electromagnetic force is not the same for all particles. The electron, for example, has a gravity to electromagnetic force ratio of only 4.537E-20.

TABLE 16
Gravity to Electromagnetic Field
Strength Ratios for Iron 5-D Triplets using Protons

| | r | R | | Seiche | | Stength |
|---|---|---|---|---|---|---|
| | Cylinder | Proton @65% | 5-D Cylinder | Completion | Seiche Count/ | Gravity to |
| Triplet | Height | cylinder rdius | VolumePa | th | EM 5-D Count | EM by triplet |
| H | 1,105 | 1,492 | 2.20E+19 | 972 | 4.41E−17 | 1.95E−33 |
| He | 2,465 | 3,328 | 2.71E+21 | 972 | 3.58E−19 | 1.28E−37 |
| Be | 3,145 | 4,246 | 1.17E+22 | 972 | 8.30E−20 | 6.89E−39 |
| Li | 3,315 | 4,475 | 1.61E+22 | 972 | 6.05E−20 | 3.66E−39 |
| c-12, Singlet O | 3,485 | 4,705 | 2.17E+22 | 972 | 4.48E−20 | 2.01E−39 |
| 0, Ne | 4,505 | 6,082 | 1.01E+23 | 972 | 9.61E−21 | 9.24E−41 |
| Ca | 5,185 | 7,000 | 2.35E+23 | 972 | 4.13E−21 | 1.71E−41 |
| | 6,205 | 8,377 | 6.91E+23 | 972 | 1.41E−21 | 1.98E−42 |
| | 7,395 | 9,983 | 1.98E+24 | 972 | 4.91E−22 | 2.41E−43 |
| | 7,565 | 10,213 | 2.27E+24 | 972 | 4.29E−22 | 1.84E−43 |
| Ar | 7,735 | 10,442 | 2.59E+24 | 972 | 3.75E−22 | 1.41E−43 |
| | 8,245 | 11,131 | 3.80E+24 | 972 | 2.56E−22 | 6.54E−44 |
| | 8,580 | 11,583 | 4.83E+24 | 972 | 2.01E−22 | 4.05E−44 |
| | 9,265 | 12,508 | 7.65E+24 | 972 | 1.27E−22 | 1.61E−44 |
| | 9,435 | 12,737 | 8.54E+24 | 972 | 1.14E−22 | 1.30E−44 |
| Fe | 9,605 | 12,967 | 9.50E+24 | 972 | 1.02E−22 | 1.05E−44 |
| | | | | | Triplet Avg. (ex H | 9.39E−39 |

A careful examination of the data reveals that the gravity scale for each of the triplets is different due to the differences in the relative radii of the triplets for each of the elements.

Cosmological constant—The cosmological constant is predicated on gravitational expansion waves emanating outward from a single point. The Axial Model includes that, measuring the cosmological constant in the context of mass (whether particles, atoms or celestial objects) gives a value of zero because the gravity waves generated outward by the synchronous flow of the trapped energy also flow back inward through the centerpoint, equal and opposite to the original outbound waves. Gravity waves generated on scales larger than those required for mass are not limited by the speed of light and have organizing forces on the scales of planets and galaxies.

Black holes—The 6/4 axial structure of the atom appears to be the same for the black hole. In a black hole, the energy is enormous since the black hole is operating as a single-particle system with unified flow and large high-density trapped paths, generating extremely strong electromagnetic fields and gravity. High-density completion paths can form on large levels, as long as the 4-D path returns to the original seiche and there is sufficient energy and lattice density for particle growth.

A black hole operates as a single-particle system with unified flow and a large completion path (like a giant neutron), generating extremely strong unified fields and gravity. In contrast, a planet or any non-homogeneous material acts as a multi-particle system and the gravitational effects are not as unified since it acts as many incoherent/incompatible small systems.

Since the high-energy completion path does not interact with standard photon energy levels, there are virtually no chaos effects of conventional temperature; therefore a black hole is cold and the completion paths are dark. In any system, the higher the uninterrupted energy flow level, the “colder” the system. Energy is taken in and released by the remaining eight cones structures in the black hole system at more conventional levels and vectors.

Neutron Star Collapse—The Model describes the real field generated by the neutron to maintain its volume in a neutron star while lattice spacing maintains the structure until the completion paths are broken. As a neutron star collapses, it releases neutrinos and high-energy photons causing the “second explosion” for larger mass stars. Large amounts of energy can be released while leaving plenty of energy for the formation of the black hole.

As shown by the model, the completion path is a narrow transfer of energy from point-to-point. In the context of the star, most of the energy and matter could be blown away and still yield a massive black hole. This is the source of the black hole information paradox.

As a neutron star's energy is transformed into a black hole structure, the strength of the gravity waves can be many times that of the original star, using only part of the original energy. The complex flow of the black hole torus is not visible conventionally and when undisturbed (unfed), and would not emit light. This may explain a black hole's occasional dark or inactive appearance. The black hole, consistent with a neutron structure, would have no apparent charge. The immense electromagnetic and gravity fields would follow the same rules as any other particle.

Dark matter—Projecting forward with the model, there are several possible sources for “missing dark matter”. First, the calculations for mass gravity need to be adjusted to account for real fourth, fifth and sixth dimensions. Second, the gravitational scale relative to the electromagnetic scale is not the same for all particles. Calculations and analysis reveal that hydrogen has a higher gravity value per proton than does iron. Third, within the cosmos there are scales of organizational waves larger that those required for mass, possibly providing a hidden level of organizational force to stellar matter.

### EXAMPLES OF THE AXIAL MODEL AND ITS APPLICATIONS

The following examples are processes for constructing embodiments of the invention. Those skilled in the art should, make various changes in the specific embodiments that are disclosed and still obtain a like or similar result without departing from the spirit and scope of the invention. These examples are not to be construed in any way as imposing limitations upon the scope thereof. On the contrary, it is to be clearly understood that various other embodiments, modifications and equivalents thereof may, after reading the description herein, suggest themselves to those skilled in the art without departing from the spirit of the present invention and/or the scope of the claims.

The source of narrow-spectrum coherent light described herein may include a laser, diode laser, LED or spectrally filtered lamp. Polarization can enhance the interaction of the light with the target because the frequencies become phase aligned. In the case of oxygen, with its paramagnetic structure, phase-aligned delivery more closely resembles the structure of energy it provides to a redox reaction. Non-coherent light includes white light and heat-generated electromagnetic radiation.

### Example 1

### Construction of the Axial Model

The novel Axial Model is constructed using a series of geometric relationships derived from solutions to a limited series of equations. The Model describes the mathematical rules for the organization of fields and the sequential energy transfer rules to describe forces within the atom. These rules and geometries provide utility for accurately predicting the deterministic position of particles and the propagation of forces from within the atom.

The model provides the underlying metric for the atom comprising 15 four-dimensional axes converging to a single six-dimensional centerpoint (FIGS. 1 and 2). Using this axial structure the Model further divides the organizational structure into axial triplets. Within each triplet, energy naturally and sequentially transfers between high-density lattice sets of four-dimensional spaces in what are defined as completion paths, describing all particles with mass.

The Model axially aligns neutrons, protons and electrons. Particles are added in sequence based on achieving the next highest energy configuration. The Model shows how to increase mass in layers of ten potential proton and neutron positions per level within the 6-D structure (FIG. 23). The model describes the structure of a photon for use in developing applications to match photons. The model can be adjusted to describe the tightening metric associated with bonding energy and energy transfer.

The model also defines the structures for emitting and absorbing photons. The model also makes the structure of symmetry understood and thereby provides the rules for symmetry breaking.

The model may be constructed on a physical or mathematical basis as follows:

Initially, high-density lattice set solutions derived from the equations (3) through (5) are chosen. The high-density radius solutions to the small radius “r” in the spindle torus equations (7) provided on Table 8 are then applied. The lattice set solutions are reviewed for possible set solutions to the substructure of the particle being modeled. The substructure radii solutions divisible by 5 create minor confinement structure while solutions divisible by 3.4 support major confinement particles.

In order to model additional particles within a triplet, the metric has to be tightened to the point where the new particle radius is sufficiently tight to allow maximal distance energy to be matched and transferred. The scale is determined by the radius set solutions set forth in Table 14. The scale of the neutron should be represented as 15.2% smaller than the proton on the same triplet. The differences in energy required tightening the metric on one side of the equator relative to the tightening to add an additional neutron or proton guides the placement of the next particle.

Each proton is modeled as the same mass despite having large differences in relative radius. The proton for iron has a radius of 9605, relative to the lattice radius of 1105 for hydrogen. Both particles have a seiche count of 108 points and therefore both are measured as having the 3-D mass of a “proton”.

The model can then be rendered in several ways:

As a flat 2-D model that highlights the levels and proton/neutron counts and positions, then provides the axial orientation of the particles (FIG. 27).

As a 3-D model, rotating the model through time to create a spindle torus, highlighting the structure of fields and particles in a more realistic context displaying positive space (FIG. 26).

As a 6/4 model showing the accurate depiction of particle positions and forces, including chiral fields, magnetic moment, charge, force, electromagnetic field and gravity generation.

As a mathematical or computer model for simulation and prediction of particle interactions.

Other features of the Model include identification of the structure and light signature of individual particles and triplets, field structures and particle geometry, which may be used to facilitate accurate modeling and manufacturing of drugs, chemicals and compounds. The Model is also useful for modeling elemental bonds and energy; gravity; computer processing and memory; photon absorption, emission and energy release variables; stellar phenomenon; and for calculating target frequencies for altering resident energy.

### Example 2

### A Method for Changing Resident Energy of Atoms

The Axial Model provides a method for targeting change (increasing or decreasing) in resident energy within an individual particle, axial triplet or, over time, increasing the resident energy in the entire atom. A narrow spectrum light applied consistently over a period ideally ranging from of several seconds to 30 days or more to the atom at a frequency slightly higher (1 to 3 nm) than the target spectra but remaining below heat levels, increases the entire atom's resident energy through equilibration. Frequencies slightly lower than the target will reduce the resident energy of the target frequency due to metric loosening and weaker seiches draining stronger ones. Tuning the frequency may be required as the resident energy of the particle changes, changing the target.

Energy for metric tightening can be delivered to the target by adding a single narrow frequency band of electromagnetic radiation, ideally from a single direction for an extended period of time. Several frequencies of light can be delivered sequentially. Disturbance from a burst of electromagnetic radiation can release resident energy spontaneously. Also neutral resident energy of some compounds can be tapped using nominal amounts of energy.

Secondary frequencies of atoms are chosen in order to add resident energy to continue metric tightening where it has already occurred. In the case of oxygen, the highest excited state emission frequencies are 844 nm and 1302 nm. These frequencies are least likely to contribute to energy buildup in the atom when it is irradiated because the seiche structure is the loosest within the atom. Also, where possible, the least mass atom is selected for change as it takes less energy to tighten the metric of a lower mass atom.

An example of the increase in resident energy for strengthening of biological processes can be demonstrated using the nematode C. elegans as a DNA aging model. Telometric shortening has been associated with normal aging and is thought to be related to lifespan so that delayed shortening may increase lifespan. The telomere-aging model is also applicable to well known yeast, fruit fly and higher organisms.

The following procedure, illustrated with C. elegans, is expected to show that worm lifespan can be increased, up to at least 50%, as indicated by the number of worms reaching maturation and which have increased telomere length compared with controls. Additionally, it is expected that any increase in telomere length will be transmitted into progeny.

A transgenic strain of C. elegans cultured, washed and filtered through 0.5 μm mesh to allow L1 Larvae to pass through. The worms are aliquoted onto new NGM plates and immediately exposed to cold LED laser light for a period of 7 days in an otherwise dark room. Several frequencies are tested to bracket the results, including 490 nm, 640 nm, 610 nm, 655 nm, 720 nm, 840 nm and 950 nm. The LED light is applied at less than 0.05 mW with high divergence to the culture until increases in number of mature worms are observed. Alternatively, coherent narrow band wavelength light may be used.

This method will also be applicable to changing the resident energy of foods to enhance growth or nutritional characteristics and to changing resident energy in compounds for application in healing. Additionally, enhancing bonding in chemical and drugs, charging an atom subsequent to release of resident energy in a burst upon application of disruptive non-coherent electromagnetic radiation, applying spectral energy to stem cells to stimulate differentiation, changing resident energy of individual elements within DNA to enhance bonding and replication and identifying target elements for reducing the resident energy of cancer cells are also within the scope of the invention and represent only a few of the many possible applications of the new Axial Model.

Singlet oxygen damage within DNA is one of the most serious negative factors in cell aging. Singlet oxygen is structured on only four 6/4 axes instead of the standard oxygen on five axes, making it magnetic and highly reactive in disrupting normal DNA replication. The prime frequency of the rouge neutron/proton is 634 nm. A spectral frequency of 615 nm applied at low power (less than 5 mW) for 15 minutes to 24 hours/day from a single direction reduces the energy of singlet oxygen bonds and at the same time adds resident energy to productive oxygen double bonds in the 604 nm to 616 nm range.

### Example 3

### Single-handed Photons

The Axial Model discloses that each particle is made up of three completion paths and that these paths share the same sequence of seiche transfer, resulting in an individual particle having only one chiral sequence and therefore only one helical rotation of the photon that particle generates.

Axially symmetric particles have opposite chirality; that is, a proton on one side of the atom rotates in the opposite direction as the proton axially opposite in the triplet cone. These structures emit two different handed photons. Further, depending on the geometry, two opposite protons are likely to have different frequencies because the metric is tighter on one side of the equator from the other.

In an atom with complete triplets (two neutrons, two protons and two electrons) the tendency is to randomly release left-handed or right-handed photons because the 6/4 axes are self-referencing across the centerpoint. Disturbances that release just one photon are just as likely to release left-handed or right-handed photons. This predictive limitation is further compounded in a group of atoms where the statistics of handedness approach 50:50.

Single-handed photons can be elicited from single protons and electrons within asymmetric atoms such as lithium, boron, nitrogen, fluorine and sodium. The Model provides insights for apparent violations of symmetry rules. Applying specific secondary spectral wavelength (610 nm or 460 nm) adds photons to the completion sets of secondary particles within the atom. That energy then generates equilibration throughout the atom, and a weak stimulus will release a photon with known chirality at the 671 nm frequency from the outermost proton within the lithium atom (FIG. 26). More massive asymmetric atoms require significantly more energy to be added to the atom in order to release a photon from the lone proton.

The following is an example of a procedure for deterministic production of a single-handed photon. A single atom of lithium can be added as an impurity to a zero phonon crystal of known properties, such as xenon or krypton. With a lithium atom bound in place, a coherent narrow band laser can charge the atom to emit single-handed photons. A laser operating at 610.5 nm is focused on the sample from a single direction. Once the laser has added energy to the atoms a brief burst of 413 nm photons is applied to disturb the completion path at 671 nm whereupon single-handed photons are released. The rate of input of 610 nm light is modulated to speed up and slow down the release of 671 nm photons. The 671 nm photons are subsequently measured for chirality and will be homogeneous.

In the event that multiple atoms are added to the crystal, ratios of left versus right-handed photons can be established for the individual crystal to create a light signature for the emitting source. In more complex atoms, higher levels of energy are required to charge up the entire atom; however, another particle within the triplet can be targeted, reducing the required energy.

One may also generate single photons using a third secondary intensity wavelength to stimulate the release of individual photons as ones and zeros for computer applications and telecommunications. Light can be added to an atom as a storage device whereupon secondary photons can later be released in a cascade effect, stimulated by bursts of coherent and broad-spectrum electromagnetic radiation.

### Example 4

### Redox Reaction Stimulation

The Axial Model illustrates that the underlying mechanism for oxidation is based on matching of axial fields and transfer of energy from the more excited system to the less excited system. The Model describes the position and spectral character of outermost particles that can potentially be bound and provides a means to match the chiral fields of the atom by addition or subtraction of appropriate wavelength energy.

In chemical reactions, the same approach can be taken once the axial parameters of the bond have been determined. Frequencies of energy can be specifically targeted to tighten and loosen the respective atoms to be bound. Those frequencies can be applied until a reaction is substantially complete.

The spectral energy of any element in a chemical reaction can be simulated to act as a catalyst to increase or speed up product output (or slow down or reduce product output). Further, using the Model, specific frequencies can be determined to have specific utility in a given reaction and intensity can be adjusted to match reaction requirements. Preferably, energy is added from a single direction with individual frequencies delivered sequentially to complete reactions with intermediate steps.

Higher yields of chemical redox reactions have significant value in commercial processes. Redox reactions in commercial applications or biological systems can be limited in two ways: (1) by running out of available atoms to complete a step in the reaction or (2) they are limited by the availability of the appropriate energy exchange between available atoms and/or catalysts. While in theory, all atoms of Type A should combine with all atoms of Type B; the reaction often results in low product yield. According to the disclosed Axial Model, the energy requirements for the oxidizing agent outpace the energy that can be supplied by the reduction agent. The Model includes that this energy can be added back to the reaction using multiple frequencies of photons associated with the reductant to substantially complete the reaction.

### Example 5

### Increasing ATP Output

An important biological redox reaction is electron transfer mediated by ATP. Applying spectral frequencies associated with oxygen can significantly enhance the ATP cycle. Biological targets such as cells or tissues may be irradiated concurrently or successively at power levels over periods of time determined by the completion of the reaction. In wound healing this effect is most pronounced following hypoxia where the ATP cycle must be re-stimulated. Different tissues will require frequency, intensity and duration adjustments based on their structure and environment.

As an example, it can be shown that the energy from oxygen required for completing the ATP energy cycle within the cell can be substantially augmented using photons associated with spectral oxygen wavelengths at low power.

The equipment is comprised of narrow spectrum coherent light using a laser to match the relative intensities of oxygen spectral frequency emissions (typically ±5 nm @ 50%) closely matching spectral intensity as cited by the National Institute of Science and Technology (NIST) Atomic Spectral Database.

Narrow spectrum wavelengths of coherent light will be applied individually to the cells. For example, the nanometer wavelengths associated with oxygen that provide most of the non-ionizing spectral energy, avoiding destructive UV wavelengths are: 595, 604, 615, 634, 645, 700, 725, 777, 822, 844, 926, 1130, 1168 and 1316 nm. The spectral energy of singlet oxygen can be delivered using light without the risk of destructive singlet oxygen bonds.

The intensity of the laser output to the target can be modulated. The light can also be phase aligned using a polarizing filter, further facilitating the reduction of applied power to the target.

As an example, HeLa cervical cancer cells are cultivated and prepared for exposure in standard petri dishes with test and control groups each receiving standard Vitacell growth medium (ATCC) and appropriate antibiotics. The control cells are placed in a hypoxic chamber where the ambient oxygen level is already reduced to 30% of normal. The control cells are harvested at 20-minute intervals. Cell death is monitored until substantially all the control cells are dead (>95%). The cells are stained with Tripan Blue to determine live/dead count.

The test cells are placed in the chamber and the light array is turned on at a power level tuned to deliver 5×10³ J/m². Output is modulated to optimize the reaction parameters.

The results of the experiment will show that the test cells survive when they are exposed to elemental oxygen frequencies that enhance and maintain the redox reaction in the otherwise sub-minimal oxygen environment.

Alternatively, selected frequencies based on the desired product may be applied serially; or energies associated with specific axial bonds may be delivered sequentially to control the handedness of the product for deterministic production of mirror compounds. Frequencies that lead to undesired bonds should be avoided. A manufacturing processes where light is applied to a redox reaction to enhance or retard specific bonds may also be developed such as applying elemental energy to a continuous or batch redox process.

Biological and chemical reactions often require specific levels of energy to complete intermediate reactions. The Model shows how the strengthening or shifting of a field can match directions or radical helicoids for bonding purposes. Using the spectral frequencies associated with the individual atoms to be bound and analysis of each atom's spectral frequencies, the bonds and energy states required for the electromagnetic fields to interact most productively may be determined.

In the case of oxygen and hydrogen, a number of frequencies are closely matched indicating that the radical helicoids share similar geometry, especially at 102 nm and 97 nm. By increasing hydrogen's energy levels to n=3 and n=4 to match the frequencies available from oxygen, bonding is facilitated and water is produced.

### Example 6

### Adding Resident Energy to Gold

Excess resident energy can be stored within an atom and subsequently released using spectral wavelengths associated with isolated spectra or orphan wavelengths of an element. In the case of gold, The smallest clusters of gold atoms are produced in the following manner: 1 gram is dissolved in a mixture of one part nitric acid with three of hydrochloric acid, the acid is removed, the sample is treated with sodium chloride, washed, and dried to a powder using known techniques. The sample is placed in a cool dark chamber and irradiated at 769 nm, at intensity levels ideally below the level that generates no more than a 20° C. rise in temperature vs ambient over a period of time determined by the application, but may be as long as 30 days in order to achieve sufficient energy for spontaneous release. The sample can be measured for low level output of additional gold spectra above and below 769 nm as the sample equilibrates to its original ground state. The sample will release stored energy when stimulated with an electromagnetic flash. The additional stored energy will be released spontaneously.

The gold sample also will slowly release energy when exposed to weak acids. The product can be ingested, implanted or injected for nutritional and therapeutic purposes.

### Example 7

### The Reduction of Glucose Using Glucose Oxidase (GOx) and Light

The purpose of this experiment was to determine if application of visible light could affect the rate of reaction of a redox reaction. The reaction chosen was the action of glucose oxidase on dl glucose, which converts glucose to hydrogen peroxide and gluconic acid.

The reaction chamber was a standard Yellow Springs Instrument oxygen meter Model 5300. This instrument employs a platinum oxygen electrode, which detects the partial pressure of oxygen in the reaction medium. The reaction medium contained buffered phosphate solution, dl glucose and glucose oxidase. All reagents were purchased from the Sigma Company. A commercial halogen light was used to irradiate the reaction chamber.

The reaction chamber, containing phosphate buffer and glucose oxidase was heated to 37 degrees C. and allowed to establish a baseline. Ten micro liters of 25% glucose was introduced into the chamber to start the reaction. After a steady reaction was produced, indicated by a consistent reaction rate slope, the chamber was irradiated with white light from a halogen light source at a distance of 6 inches from the chamber. The light was turned off and on at various time intervals.

Almost immediately after application of the light the reaction is stopped, as evidenced by a flat trace on the recorder. The inhibition lasted about 2 minutes before the reaction resumed the original rate. After approximately ten minutes the chamber was again irradiated with the light. Inhibition occurred again and lasted about the same time. Repeated experiments showed the same results. Inhibition could be prevented by increasing the concentration of the reactants.

As the model predicted, non-coherent light energy inhibits the glucose redox reaction. The inhibition is temporary and appears to be related to the radiant energy and the concentration of the reactants.

A second experiment can be performed to demonstrate that coherent light enhances the reactive potential of Glucose oxidase and glucose. Samples are prepared and allowed to develop a standard rate of reaction at constant temperature and pH. The reaction rate is measured for ten minutes. Once the baseline standard is established, the sample is irradiated with laser light as described in Example 2. Light is applied from a single wavelength laser appliance at individual wavelengths including 595 nm, 700 nm and 962 nm, which are associated with oxygen adding energy to carbon. The carbon is energized to release an electron and proton while oxygen is de-energized to accept the hydrogen proton. With laser application of the individual wavelengths, the appliance can be energized to add energy until the reaction is enhanced to provide increased reaction products and/or increased reaction rates.

Applications, Advancements and Alternative Embodiments of the Invention

The Axial Model confirms the unification of the major theories in physics today. The Axial Model is based not on a new algorithm; rather, it is based on defining the fundamental physical structure of the atom in six-dimensions thereby providing the structure and causality for all atomic events. Thus, the Model is a novel unification of the major theories in physics and does not introduce concepts or algorithms incompatible with such theories.

The Model definitively demonstrates that spontaneous matter formation is based on convergence of six directions/dimensions according to simple rules. The Model confirms that mass is measured in three dimensions, that energy transfers within a four-dimension axial lattice structure, that particles and their fields are constructed using triplet sets of four-dimensions (five dimensions total per triplet) and that the entire atom is based on a six-dimensional metric structured around a naturally occurring 6-D centerpoint.

The Axial Model is consistent with experimental data and provides natural explanations to a many phenomena that otherwise appears inconsistent with current theories in physics. In terms of energy transfer, the Model is consistent with current four-dimension space-time models in its use of four dimensions to describe force transfer in detail within the six-dimension atom.

The Axial Model illustrates the natural structural reasons for particle scales with no compromises or missed steps from the proton down to the structure of a photon and a single lattice point, a scale of 5.05E-21 versus the proton and consistent with the scale of string theory. This solution is based on using the natural radii of high-density lattice sets as tube radii of spindle torus equations, creating particles of definable scale. The Model also predicts numerous other particles that have yet to be discovered (e.g., the electron quark).

Another important feature of the Axial Model is that it provides the machinery for the excitation states of electrons in hydrogen without requiring an increase in the electron radius, an unworkable construct when attempting to Model the excitation of many-electron atoms. It also includes why a photon is both a particle and a wave and how photon energy is absorbed and emitted within mass. The Model also indicates the determinable positions of electrons.

The Model, however, challenges one set of historic conclusions by reassigning the conclusions drawn from Rutherford-type scattering experiments. Consistent with observations, the Model adds that all particles within an atom are tied to the centerpoint and can be measured through the centerpoint; however, all that defines mass is not located at the centerpoint.

The Model discloses the mechanism for “resident energy” within the atom and the tools to manipulate resident energy levels. The Model provides the method for changing the complex energy levels (foul through six dimensions) within individual atoms using specific spectral wavelengths applied to atoms, molecules, elements and compounds. Energy and structure can be manipulated in both organic and inorganic systems. Target description and methods for manipulating molecular bonds and the synthesis of compounds are readily identified through use of the Model. The Model is unique because it is predictive of results that are amenable to resident energy manipulation within the individual atom.

In cells, changing resident energy levels of atoms within DNA changes electromagnetic field generation, bonding strength, production of proteins and cell replication. The higher the level of resident energy that can be added for example, to carbon, nitrogen, and other atoms, the stronger the associated bonds. The stronger the organization of replication fields and the less likely bonds are to break.

The Model has tremendous utility in that it defines the structure of the atom and yields predictive information about atomic structure, especially in the context of interaction with other atoms. Architecture and energy relationships within the electron orbit as defined by current physics theories have limited use in biology, chemistry and biotechnology applications, however, there are features of the Model that relate to sciences other than physics; for example, the ability to manipulate resident energy in chemical reactions and covalent bonds.

The Model reveals that when atoms interact, there are two exchanges: first, atoms contribute organization to potential interactions such as bonding, and second, atoms transfer energy from the higher to lower energy atoms. Organization is provided in the nesting of electromagnetic fields according to rules described by the Model. The Model reveals how these alignments are facilitated for the enhancement of atomic interactions, including redox reactions.

The Axial Model also shows that the axial orientation of the neutron, proton and electron provides the underlying structure for chiral fields and that the atom can be aligned in a manner to facilitate the generation of single-handed photons from an atom. These applications relate to computers that operate on photons instead of being limited by electrons, information storage devices on a photon level, telecommunications improvements and enhanced encryption.

An important aspect of the invention is the ability of the Model to not only describe the structure of the atom and particles, but also to provide a tool for determining the proactive changes that can be made to the structure for the purpose of predicting and predict the material outcomes. The Model also includes how to manipulate the six-dimension energy levels within atoms using low energy for a variety of useful applications from medicine to computing. Brief summaries of selected applications are provided below.

Identification of atom structure and constituent particles—The disclosed Model provides a convenient and accurate method to determine the size of particles and to place measured forces within a context in the atomic structure. The Model provides specific guidelines and rules for the scales of bonds and the tightening metric as well as bond formation and bonding angles within elements, crystals, molecules and compounds.

Math sets and structures are provided for analyzing and determining the scale and character of particles and forces that have not yet been experimentally identified. The Model is also useful for determining the chirality of each particle and radical axis structure of individual atoms.

Changing resident energy levels—The Model provides a method for changing (increasing or decreasing) the energy flow within an individual atom, particle or axial triplet and, over time, increasing the resident energy of the entire atom. The Model also provides a method to change the bonding axes associated with molecule formation and crystal formation. The Model includes how to measure changes in complex energy within atoms even though it is not visible conventionally. The Model also guides metric tightening and loosening with photon energy

Production of single-handed photons—Light sources can be refined to emit left-handed or right-handed light based on elemental particles emitting solitary photons of predictable chirality. Currently, light is polarized through filters in bulk quantities. The ability to emit left and right spin particles at the single-photon level will significantly improve communication, computer performance and encryption.

Further, the Model also includes which atoms have double-matched particles, that is, particles that are axially positioned such that the photon characteristics for two axially symmetric particles are matched, except for chiral direction. These axial atoms are useful in duplicating photons accurately for computing and telecommunications.

Improving memory storage—Manipulating resident energy within individual atoms using photons will drive new computer memory and storage devices. Providing timed energy or obstruction enables gates and switches at the atomic and molecular level. It will improve nano-technology and reduce the constraints on transmission of information on small scales currently affected by field effects due to electrons.

Minimizing flow obstruction—Superconductors operate best at extreme low temperature because of unobstructed flow paths. The higher the resident energy, the more resistant the element is to flow disruption. Higher flow integrity will lead to higher temperature superconductors.

Changing atomic structure—The Model includes that the structure of an atom is quantized on many levels and can be tightened, increasing the complex energy of the atom and allowing for new elements to be formed based on addition or subtraction of energy to or from the atom.

DNA amplification—Amplification of DNA by the polymerase chain reaction can greatly be simplified using light, sequentially or concurrently adding specific spectral wavelengths for increased flow followed by release with a burst of appropriate multi-wavelength light. This enables: (1) decoding of DNA at the atomic level (C, N, O, P, H); (2) changing the resident energy within DNA atoms allowing manipulation/enhancement/repair of DNA and gene function, differentiation of stem cells, improvement of chronic conditions and design of targeted therapies in biological systems and (3) determining the architecture of genes and gene processes enabling enhancement of specific genes and regulation of selected gene products.

Drug discovery—Some of the applications of this technology, include: (1) a deterministic Model for atoms, molecules and compounds for computer simulation of interactions; (2) methods for changing resident energy within elements, molecules and compounds for effective therapeutic results; (3) a perturbation-free physics model for elements, molecules, crystals and compounds; (4) a method and applications for deterministically creating left-handed and right-handed handed bonds; and (5) a method and applications for adding energy to redox reactions to promote specific outcomes on a more timely basis.

Molecular synthesis—The Model allows determination of the complex nature of protein structures, making the manufacture of enantiomers (mirror compounds) more predictable and efficient and also providing advanced modeling of potential compounds and drug targeting. Using the methods described, significant improvements can be made in the design and synthesis of drugs by selectively choosing bonds to enhance during production. The design of drugs or chemical compounds and a means to predictably influence redox reactions become possible.

Further enhancements are also possible with the Model, including, changing the angles and available helicoid alignments to align with those predicted by the Model and using wavelengths at angles ideally suited to a bond (e.g., carbon in diamond form). There are many practical applications of this technology in medicine, chemistry, biology and material fabrication.

In a particular aspect, the atomic structure provides information valuable to the construction and utilization of atoms with specific utility. This Model will significantly reduce time and expense to predict and prove new particle properties.

Medical therapy—The atomic Model provides a tool for evaluating disease and chronic conditions as a result of overactive or weakened resident energy within atomic functions in the host. The Model will provide a means for determining the frequency(s), sequence(s) and duration(s) of single/multiple wavelength(s) of light that can be added to selected atoms in an effort to enhance the host or weaken the disease. This energy can be applied directly to the affected area or imparted to other atoms, molecules and compounds to be delivered to the site. The Model will assist in modeling how disease takes hold and how to treat it.

A device can be created to simulate elemental energy (e.g., oxygen) has utility to stimulate reactions and control interactions of elements. For example, delivering the excited states frequencies of oxygen to cells can immediately provide energy required for the ATP cycle and can initiate the production of protective proteins and angiogenic factors.

The model may provide insight to brain function on a resident energy basis because the most accurate brain scans measure electromagnetic field activity. According to the model, memory and retrieval can be affected by low energy transfers within atomic field structures storing and releasing resident energy.

Foods and nutrients—The Model demonstrates that by adding or subtracting complex resident energy to or from food (feeds, nutrition, vitamins, mitochondria supplements and ingestible ingredients/compounds/molecules/elements). The field effects of atoms within the food can be proactively altered to supply desired nutritional effects to living cells and systems. These effects can be selectively used to increase general wellness and also treat specific conditions. The addition of resident energy can be provided also by a surrogate and then delivered to the target through drugs, implants, topically applied compounds and ingestibles.

Nuclear modification—The Model includes that by adding energy at appropriate wavelengths under appropriate conditions of trapped energy path flow and obstruction, the resident energy can be increased and the metric tightened, leading to the formation of new particles within atoms or the deterministic formation of new atoms. The intersection of helicoid axes and the release of ultra-weak photons along the helcoid induce formation of new centerpoints. Photon wavelengths of appropriate scale increase or decrease the atom's energy, and the timely introduction of flow disruption (through application of heat, broad spectrum electromagnetic waves or targeted monochromatic light) creates changes in the metric such that the flow is constructively altered to manipulate particles.

Fuel efficiency—Fuel cracking/synthesis can be enhanced by changing levels of resident energy, bond angles or flow release parameters. Modeling combustion and the rapid release of energy from the atom as a rapid sequential release is essential to the development of more efficient fuel systems. The Model also includes how resident energy within atoms and bonds can be increased and released on a controlled basis or in a burst. Improved combustion and reconditioning old fuels is possible with select use of spectral wavelengths.

1. A method of temporarily inhibiting oxidation of glucose in the presence of glucose oxidase and oxygen, comprising:
irradiating a glucose/glucose oxidase solution with non-coherent light for a period of time sufficient to temporarily inhibit the oxidation of glucose.

2. The method of claim 1 wherein the non-coherent light is low-energy.

3. The method of claim 2 wherein the non-coherent light is white light.

4. The method of claim 3 wherein the white light is from a halogen light source or heat generated electromagnetic radiation.

5. The method of claim 1 wherein the non-coherent light source is about 6 inches from the glucose/glucose oxidase solution.

6. The method of claim 1 wherein the inhibition is at about 37° C.

7. The method of claim 1 wherein glucose and glucose oxidase are comprised within a cell.

8. A method of at least temporarily increasing the rate of oxidation of glucose in the presence of glucose oxidase and oxygen, comprising:
irradiating a glucose/glucose oxidase solution with narrow spectrum coherent light for a period of time sufficient to at least temporarily increase the rate of glucose oxidation.

9. The method of claim 8 wherein the coherent light comprises a non-ionizing narrow band spectral energy wavelength selected from the group consisting of 595, 604, 615, 634, 645, 700, 725, 777, 822, 844, 926, 926, 1130, 1168 and 1316 nm.

10. The method of claim 9 wherein the spectral energy wavelength is 844 nm or 1302 nm.

11. The method of claim 8 wherein the glucose and glucose oxidase are comprised within a cell.
